(19) United States
(12) Patent Application Publication
(10) Pub. No.: US 2022/0283161 A1
(43) Pub. Date: Sep. 8, 2022

(54) METHODS AND COMPOSITIONS FOR HEPATITIS C VIRUS (HCV)

(71) Applicant: The Johns Hopkins University, Baltimore, MD (US)

(72) Inventors: Valerie Kinchen, Baltimore, MD (US); Guido Massaccesi, Baltimore, MD (US); Stuart C. Ray, Baltimore, MD (US); Andrea Lynn Cox, Baltimore, MD (US); Justin R. Bailey, Baltimore, MD (US)

(21) Appl. No.: 17/630,104

(22) PCT Filed: Jul. 24, 2020
(86) PCT No.: PCT/US2020/043429

§ 371 Date: Jan. 25, 2022

Related U.S. Application Data
(60) Provisional application No. 62/878,631, filed on Jul. 25, 2019.

(51) Int. Cl. G01N 33/576; A61K 39/29; C12N 7/00

(52) U.S. Cl. CPC G01N 33/5767; A61K 39/29; C12N 7/00; A61K 2039/575

(57) ABSTRACT

Provided herein are, inter alia, methods, compositions and kits for HCV antigen and vaccine design. Preferred methods include measuring neutralization of HCV pseudoparticles (HCVpp) by antibodies specific for an HCV in the biological sample, generating a neutralizations profile of each biological sample; deconvoluting the HCV-specific neutralizing antibodies by generating reference antibody neutralization profiles; correlating the reference antibody neutralization profiles to the biological sample's neutralization profile; and, identifying the HCV neutralizing antibodies.

### PRIORITY CLAIM

This application claims benefits of priority to U.S. Provisional Application No. 62/878,631 filed Jul. 25, 2019, the entire contents of which are incorporated herein by reference.

### STATEMENT AS TO FEDERALLY SPONSORED RESEARCH

This invention was made with government support under grant AI127469 and AI088791 and awarded by the National Institutes of Health. The government has certain rights in the invention.

### BACKGROUND

New compositions and methods for a vaccine for hepatitis C virus (HCV) are needed.

### BRIEF SUMMARY

Provided herein are, inter alia, methods of identifying Hepatitis C virus (HCV) neutralizing antibodies.

Preferred methods include measuring neutralization of HCV pseudoparticles (HCVpp) by antibodies specific for an HCV in the biological sample, generating a neutralization profile of each biological sample; deconvoluting the HCV-specific neutralizing antibodies by generating reference antibody neutralization profiles; correlating the reference antibody neutralization profiles to the biological sample's neutralization profile; and, identifying the HCV neutralizing antibodies.

In embodiments, the method comprises that the neutralization profile comprises a ranking of relative neutralization of each HCVpp by each reference antibody or biological sample.

In further embodiments, the reference antibody neutralization profiles are added in various proportions to generate an array of possible combined antibody neutralization profiles.

In examples, the methods herein provide that a specific combined reference antibody neutralization profile be correlated with each plasma neutralization profile to identify the proportion of each reference antibody contributing to the neutralization profile of the biological sample.

In embodiments, the methods herein further comprise identifying HCV epitope specificities for each neutralizing antibody. Also provided by the methods herein, the neutralization profiles identify individual antibodies which bind to distinct HCV epitopes or are cross-reactive to related HCV epitopes.

The methods provided herein also comprise isolating the HCV neutralizing antibodies.

In further embodiments, the method comprises a high throughput method. For example, the method can further be a high throughput method. In aspects, a high throughput method may refer to an assay which provides for multiple candidate agents, samples or test compound to be screened simultaneously. As further described below, examples of such assays may include the use of microtiter plates that are especially convenient because a large number of assays can be carried out simultaneously, using small amounts of reagents and samples. The methods are easily carried out in a multiwell format including, but not limited to, 96-well and 384-well formats as well as automated systems.

In embodiments, provided herein is a vaccine comprising a polypeptide having an Hepatitis C virus (HCV) epitope which induces an HCV neutralizing antibody, said antibody identified by the methods described herein.

For example, the vaccine comprising an HCV epitope which induces an HCV neutralizing antibody, and the antibody is identified by obtaining a biological sample from a subject having been infected with HCV; measuring neutralization of HCV pseudoparticles (HCVpp) by antibodies specific for HCV in the biological sample; generating a neutralization profile of each biological sample; deconvoluting the HCV-specific neutralizing antibodies by generating a reference antibody neutralization profile; correlating the reference antibody neutralization profile to the biological sample's neutralization profile; and, identifying the HCV neutralizing antibodies.

Also provided herein is an isolated hybrid cell producing a Hepatitis C virus (HCV) neutralizing monoclonal antibody identified by the methods described herein.

In other examples, methods of treating a subject infected with a Hepatitis C virus (HCV), comprising administering to the subject a therapeutically effective amount of HCV neutralizing antibodies identified by the methods described herein, or the vaccines described herein.

Further provided herein are methods of identifying virus-specific neutralizing antibodies comprising, obtaining a biological sample from a subject having been infected with a virus; measuring neutralization of a virus by antibodies specific for the virus in the biological sample; generating a neutralization profile of each antibody specific for each virus; deconvoluting the virus-specific neutralizing antibodies by generating a reference antibody neutralization profile; correlating the reference antibody neutralization profile to the biological sample's neutralization profile; and, identifying the virus neutralizing antibodies. In embodiments, the virus includes adenoviruses, arenaviruses, bunyaviruses, flaviviruses, filoviruses, herpesviruses, noroviruses, orthomyxoviruses, poxviruses, papilloma viruses, paramyxoviruses, reoviruses, rhabdoviruses, retroviruses, or togaviruses.

As referred to herein, the terms “subject,” “patient,” “individual,” etc. are not intended to be limiting and can be generally interchanged. That is, an individual described as a “patient” does not necessarily have a given disease, but may be merely seeking medical advice. For example, a “patient” or “subject” or “subject in need thereof” refers to a living member of the animal kingdom suffering from or who may suffer from HCV. In embodiments, the subject is a human.

As referred to herein, the term “sample” refers to a biological sample suitably obtained for the purpose of evaluation in vitro. The sample suitably may comprise a body fluid. In some embodiments, the body fluid includes, but is not limited to, whole blood, plasma, serum, lymph, breast milk, saliva, mucous, semen, cellular extracts, inflammatory fluids, cerebrospinal fluid, vitreous humor, tears, vitreous, aqueous humor, or urine obtained from the subject. In some aspects, the sample is a composite panel of two or more body fluids. In exemplary aspects, the sample comprises blood or a fraction thereof (e.g., plasma, serum, or a fraction obtained via leukapheresis).

Other aspects of the invention are disclosed infra.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a schematic of the deconvolution method described herein. Neutralization of a panel of 19 HCVpp by 8 reference mAbs 2 or 63 plasma samples was measured, generating a neutralization profile (i.e., ranking of relative 3 neutralization of each HCVpp) for each reference mAb (“mAb rank”) and each plasma sample 4 (“plasma rank”). Points on each graph represent the ranking of relative neutralization sensitivity 5 of each of 19 HCVpp by plasma on the x-axis and a reference mAb on the y-axis. Reference 6 mAb neutralization profiles were added in all possible proportions to generate an array of 7 possible combined mAb neutralization profiles (“combined mAb rank”). The algorithm then 8 identified the specific combined reference mAb neutralization profile with the strongest positive 9 correlation with each plasma neutralization profile, thereby delineating the most likely 10 proportion of each reference mAb contributing to the neutralization profile of the plasma sample.

FIGS. 2A-2C depict images showing the selection of a reference mAb panel. FIG. 2A is a cartoon showing the main chain atoms of previously defined mAb binding residues identified by alanine scanning mutagenesis (See Materials and Methods, below) marked with colored spheres on the crystallized structure of the HCV E2 protein ectodomain (gray, ribbon), strain 1b09, PDB accession 6MEI (15), with colors modified in PyMOL, v1.8.6.2. For clarity, a maximum of 5 residues with impact on binding of each mAb are marked. Antigenic Domains/Antigenic Regions (AR) are indicated, and mAbs highlighted with the same color share multiple binding residues. Dashed lines indicate disordered region (HVR1) or part of E2 protein truncated to aid crystallization (stem region of E2). Putative AR4A and AR5A binding residues on E1 are not shown. FIG. 2B is a graph showing the neutralizing breadth of reference mAbs at 10 μg/mL measured with a panel of 19 genotype 1 HCVpp, with a threshold of 50% neutralization considered positive. FIG. 2C is an image of a heatmap showing functional grouping of reference mAbs. For each mAb, neutralization of each of 19 HCVpp was measured in duplicate, generating a neutralization profile (i.e., ranking of relative neutralization of each HCVpp) (FIGS. 10A-10C), and pairwise Pearson correlations were measured between these neutralization profiles. Circles at each intersection of the heatmap are scaled by the magnitude of the correlation (r) between the indicated NAbs. mAbs were grouped into a NAb-type if the neutralization profile of each mAb in the group had a correlation ≥0.55 with every other mAb in the group. One representative mAb (indicated with an asterisk) was selected from each NAb-type to form the final panel of 8 reference mAbs.

FIG. 3 depicts a chart showing deconvolution of samples containing known mAbs. Neutralization profiles of samples containing single reference mAbs or combinations of reference mAbs were used as input into the deconvolution algorithm. Each two-mAb or three-mAb combination included 5 μg/mL or 3.3 μg/mL of each mAb, respectively. These neutralization profiles were determined in a single experiment, with neutralization of each HCVpp measured in duplicate. Reference mAb profiles were averaged from four or five independent experiments. Values are proportion of the neutralization activity of the sample attributed to each reference mAb by the deconvolution analysis. Proportions greater than 0.10 are highlighted in gray. P values indicate the Pearson correlation of the neutralization profiles of spiked-in mAbs with a combined reference mAb neutralization profile comprising the indicated proportion of each reference mAb (see Materials and Methods, below and FIG. 9.

FIG. 4 are images depicting the concordance between plasma deconvolution and mAbs isolated from B cells of the same subject. Deconvolution of NAb-types in plasma of two subjects, C117 and C110, from whom reference mAbs were also isolated from B cells. Plasma neutralization profiles each were averaged from two independent experiments, which were each performed in duplicate. Reference mAb profiles were averaged from five independent experiments, with each performed in duplicate. Wedge sizes are proportional to the plasma response attributed to each reference mAb. **, Reference mAb detected in plasma was isolated from the B cells of the same subject; *, Reference mAb detected in plasma and a mAb isolated from the B cells of this subject are of the same NAb-type; i.e., they have positively correlated neutralization profiles and compete for E1E2 binding. P values are for Pearson correlations of each plasma neutralization profile with a combined reference mAb neutralization profile comprising the indicated proportion of each reference mAb.

FIGS. 5A-5C are images that depicting plasma NAb deconvolution predicts mAbs subsequently isolated from B cells of the same subject. FIG. 5A is a chart showing NAb deconvolution of subject C18 plasma. Plasma neutralization profile was averaged from two independent experiments. Reference mAb profiles were averaged from five independent experiments. Wedge sizes are proportional to the plasma response attributed to each reference mAb. P value is from the Pearson correlation of the C18 plasma neutralization profile with a combined neutralization profile comprising the indicated proportion of each reference mAb. FIG. 5B is a chart showing Pearson correlations between neutralization profiles and number of shared probable E1E2-binding residues, as determined by alanine-scanning mutagenesis-E1E2 binding analysis, between twelve mAbs isolated from subject C18 B cells and best match reference mAbs. C18 mAb neutralization profiles were determined in a single experiment, with neutralization of each HCVpp measured in duplicate. FIG. 5C are cartoon representations showing examples of concordance between binding residues of reference mAbs identified in plasma and three best match mAbs isolated from C18 B cells. Colored spheres indicated main chain atoms of probable binding residues, superimposed on the crystallized structure of the HCV E2 protein ectodomain, strain 1b09, PDB accession 6MEI (15), with colors modified in PyMOL, v1.8.6.2. Shared putative E1 binding residues of HEPC130 and AR4A (Y201, N205) are not shown.

FIG. 6 is a chart showing deconvolution of NAbs in plasma of subjects with subsequent clearance or persistence of HCV infection. Reference mAbs are on the x-axis with plasma samples on the y-axis. Each plasma sample is from a different subject. Plasma neutralization profiles each were averaged from two independent experiments. Reference mAb profiles were averaged from five independent experiments. Values shown are the proportion of each plasma neutralizing response attributed to each reference mAb. Proportions greater than 0.1 are shown and marked with different colors for each NAb-type, with higher values shaded darker. Plasma samples are grouped by subject outcome. Neutralizing breadth was calculated as the # out of 19 HCVpp neutralized >50% by a 1:100 dilution of plasma. P values are for the Pearson correlation between the plasma sample neutralization profile and the best fit combined reference mAb neutralization profile. Only subjects with significant correlation (p<0.05) between combined mAb neutralization profile and plasma neutralization profile are shown (Persistence n=29, Clearance n=15). Results for subjects with a poor deconvolution fit are shown in FIG. 12.

FIGS. 7A-7H are graphs depicting data focusing of the humoral response toward bNAbs and expression of multiple bNAb-types was associated with HCV clearance and greater plasma neutralizing breadth. FIG. 7A is a graph showing the Proportion of Persistence and Clearance plasma responses attributed to each NAb-type. Values are means, and error bars are SEM. *, p<0.05 by one-way ANOVA adjusted for multiple comparisons using Sidak's method. FIG. 7B is a graph showing the total proportion of Persistence and Clearance plasma responses attributed to bNAbs (AR3A, HEPC74, HC84.26, or AR4A). Horizontal lines are means, and whiskers are SD. **, p<0.01 by two-sided T test. FIG. 7C is a graph showing the number of NAb-types positive above a threshold of 0.10 for each plasma sample. Horizontal lines are means, and whiskers are SD. P=0.24 by two-sided T test. FIG. 7D is a graph showing the number of bNAb-types (AR3A, HEPC74, HC84.26, or AR4A) positive above a threshold of 0.10 for each plasma sample. Horizontal lines are means, and whiskers are SD. *, p<0.05 by two-sided T test. FIG. 7E is a graph showing the frequency of each observed bNAb combination across all Persistence or Clearance subjects. FIG. 7F is a graph depicting the neutralizing breadth of plasma samples from Persistence or Clearance subjects. (# out of 19 HCVpp neutralized >50% by a 1:100 dilution of plasma) FIG. 7G is a graph showing the correlation between total proportion of each plasma response attributed to bNAbs and neutralizing breadth of that plasma sample. R and p values calculated by Pearson's method. FIG. 7H is a graph showing the relationship between the number of bNAb-types expressed by each plasma sample and neutralizing breadth of that sample. **, p<0.01 for the trend by one-way ANOVA.

FIG. 8 is a schematic depicting a method as disclosed herein.

FIG. 9 is a flow chart depicting an example calculation of a plasma deconvolution quality of fit (Subject C117).

FIGS. 10A-10C are graphs depicting the correlations between reference mAb neutralization profiles. Examples of correlations between neutralization profiles of reference mAbs falling in the same NAb-type ((AR3A vs. AR3B (FIG. 10A); HEPC98 vs. HC33.4 (FIG. 10B)) or reference mAbs falling in different NAb-types ((AR3A vs. HEPC98 (FIG. 10C)). Points on each graph represent the ranking of relative neutralization sensitivity of each of 19 HCVpp by one mAb on the x-axis and a different mAb on the y-axis. R and p-values are for Pearson correlations. Neutralization profiles shown here were determined in a single experiment, with neutralization of each HCVpp measured in duplicate. Neutralization profiles of the eight reference mAbs representative of each NAb-type were subsequently tested in four additional independent experiments.

FIG. 11 is a chart showing the deconvolution of samples containing known mAbs at two different concentrations. Neutralization profiles were determined in a single experiment, with neutralization of each HCVpp measured in duplicate. Reference mAb profiles were averaged from five independent experiments. Values are proportion of the neutralization activity of the sample attributed to each reference mAb by the deconvolution analysis. The highest proportion for each experiment is highlighted in gray. Breadth is the number out of 19 HCVpp neutralized by >50%. P values indicate the Pearson correlation of the neutralization profiles of spiked-in mAbs with a combined reference mAb neutralization profile comprising the indicated proportion of each reference mAb (see Materials and Methods and FIG. 9).

FIG. 12 is a chart show the results of plasma samples with poor fit of deconvolution analysis. Reference mAbs are on the x-axis with plasma samples on the y-axis. Each plasma sample is from a different subject. Plasma neutralization profiles each were averaged from two independent experiments. Reference mAb profiles were averaged from five independent experiments. Values shown are the proportion of each plasma neutralizing response attributed to each reference mAb, with higher values shaded darker. Plasma samples are grouped by subject outcome. Neutralizing breadth was calculated as the # out of 19 HCVpp neutralized >50% by a 1:100 dilution of plasma. P values are for the Pearson correlation between the plasma sample neutralization profile and the best fit combined reference mAb neutralization profile.

### DETAILED DESCRIPTION

Provided herein are, inter alia, methods, compositions and kits to vaccines for hepatitis C virus (HCV). A vaccine for hepatitis C virus (HCV) is urgently needed. Currently, it is not possible to accurately assess antibody response to hepatitis C virus vaccines in animals or humans. There are hundreds of millions of people in the world at risk for HCV infection.

Development of broadly-neutralizing plasma antibodies during acute infection is associated with HCV clearance, but the viral epitopes of these plasma antibodies are unknown. Identification of these epitopes could define the specificity and function of neutralizing antibodies (NAbs) that should be induced by a vaccine. Here, the development and application of a high-throughput method that deconvolutes polyclonal anti-HCV NAbs in plasma, delineating the epitope specificities of anti-HCV NAbs in acute infection plasma of forty-four humans with subsequent clearance or persistence of HCV.

Remarkably, multiple broadly neutralizing antibody (bNAb) combinations were identified that were associated with greater plasma neutralizing breadth and with HCV clearance. These studies inform new strategies for vaccine development by identifying bNAb combinations in plasma associated with natural clearance of HCV, while also providing a high-throughput assay that could identify these responses after vaccination trials. This method can be used to assess protective responses induced by vaccines rapidly and with high resolution, which will facilitate rational design and development of HCV vaccines. The assay described herein is the only assay that accurately identifies neutralizing antibodies against HCV in plasma.

### Hepatitis C Virus (Hcv)

A vaccine to prevent hepatitis C virus (HCV) infection is urgently needed. Although recently developed direct acting antivirals (DAAs) are highly effective for HCV treatment, most individuals are unaware of their infection status, so they do not seek treatment and may continue to expose others (1, 2). Most infected persons do not have access to DAAs, and treatment does not prevent reinfection after cure (3-7). Unfortunately, HCV vaccine development has been hampered by incomplete understanding of correlates of protective immunity in humans (8, 9), and by inadequate methods for assessing antibody responses induced by candidate vaccines.

Around 25% of individuals who are acutely infected with HCV spontaneously clear the infection without treatment (10). This spontaneous clearance is associated with early development of broadly neutralizing plasma, suggesting that identification of neutralizing antibodies (NAbs) present in the plasma of these individuals could elucidate the NAbs that should be induced by a vaccine (11, 12). However, the epitope specificities of NAbs present in broadly neutralizing plasma are unknown. This plasma might contain NAbs similar to known broadly neutralizing monoclonal antibodies (bNmAbs), broadly neutralizing antibodies (bNAbs) with novel epitopes, or a diverse array of strain-specific NAbs.

The current standard of care therapy includes treatment with a combination of direct acting antivirals (DAAs), pharmacologic inhibitors of the viral NS3/4A protease, NS5A, or NS5B polymerase, with overall treatment efficacy greater than 90%. Despite this progress, viral resistance to these treatments has been observed clinically, and has been associated with treatment failure. Most infected individuals throughout the world are unaware of their infection status and may continue to infect others, and treatment does not prevent reinfection after cure. The high costs of these new therapies and the large numbers of HCV-infected individual's means the health-care system, even in developed countries, cannot afford to treat all patients. This limitation is even more pronounced in developing countries.

Available methods used to identify the epitopes targeted by polyclonal serum neutralizing antibodies have notable limitations. One such method is isolation of monoclonal antibodies (mAbs) from B cells. bNmAbs have been isolated from the B cells of individuals who cleared HCV infection, (13-17) and have demonstrated that bNmAbs designated HEPC3 and HEPC74, which target the front layer of E2, contributed to viral clearance in two individuals by driving the infecting virus to an unfit state (18). However, these bNmAbs may not fully represent the complete repertoire of polyclonal NAbs present in plasma of these subjects prior to viral clearance, and these methods remain relatively labor intensive and time consuming, making it difficult to characterize a large number of subjects. One alternative approach is measurement of serum antibody binding to peptide arrays, but many NAbs bind to discontinuous, conformational epitopes rather than linear peptides (9, 19, 20). A third approach is measurement of envelope (E1E2)-binding competition between serum antibodies and reference mAbs (21), but these assays are confounded by the abundance of E1E2-binding but non-neutralizing antibodies in plasma. These non-neutralizing antibodies may compete with bNmAbs for overlapping or adjacent E1E2 epitopes (22-27), so binding competition may not discriminate reliably between neutralizing and non-neutralizing antibodies in plasma.

Given these limitations, previous studies have not comprehensively identified NAbs contributing to neutralizing breadth of plasma or NAbs that may be associated with clearance or persistence of HCV infection.

As described herein, development and application of a high-throughput method that delineates the epitope specificities of anti-HCV NAbs in polyclonal human plasma is provided. Using this method, he majority of anti-HCV plasma neutralizing activity in individuals with subsequent clearance or persistence of HCV was shown to be attributed to a set of NAbs with known epitope specificities. Notably, focusing of the immune response toward bNAbs rather than narrow-breadth NAbs, and simultaneous expression of multiple bNAb-types was associated with greater plasma neutralizing breadth and with HCV clearance. These studies inform new strategies for vaccine development by identifying bNAb combinations in plasma associated with clearance of HCV, while also providing a high-throughput assay that could identify these responses after vaccination trials.

### Methods of Identifying HCV Neutralizing Antibodies

Provided herein are methods of identifying Hepatitis C virus (HCV) neutralizing antibodies. The method includes obtaining a biological sample from a subject having been infected with HCV, measuring neutralization of HCV pseudoparticles (HCVpp) or replication competent virus (HCVcc) by antibodies specific for an HCV in the biological sample; generating a neutralization profile of each biological sample; deconvoluting the HCV-specific neutralizing antibodies by generating a reference antibody neutralization profile; correlating the reference antibody neutralization profile to the biological sample's neutralization profile; and, identifying the HCV neutralizing antibodies.

Antibodies with certain biological characteristics may be selected as described in the Examples.

To screen for antibodies which bind to an epitope on the HCV E2 protein bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as an anti-HCV E2 antibody of the invention. Alternatively, or additionally, epitope mapping can be performed by methods known in the art. In a different method, peptides corresponding to different regions of HCV E2 protein can be used in competition assays with the test antibodies or with a test antibody and an antibody with a characterized or known epitope.

In some embodiments, antibodies can also be screen for their ability to neutralize an HCV infection. In some embodiments, neutralization of an HCV infection is based on a HCV pseudotyped particles (HCVpp) neutralization assay. HCVpp consist of unmodified HCV envelop glycoproteins assembled onto retroviral or lentiviral core particles. HCVpp infect hepatoma cell lines and hepatocytes in an HCV envelop protein-dependent matter. The presence of a marker gene packaged within the HCVpp allows fast and reliable determination of antibody-mediated neutralization. In some embodiments, neutralization of an HCV infection is based on a recombinant cell culture-derived HCV (HCVcc) neutralization assay infecting human cell lines.

The methods of identifying Hepatitis C virus (HCV) neutralizing antibodies also include a neutralization profile that comprises a ranking of relative neutralization of each HCVpp and of each biological sample. In other examples, the reference antibody neutralization profiles are added in various proportions to generate an array of possible combined antibody neutralization profiles. For example, a specific combined reference antibody neutralization profile is correlated with each plasma neutralization profile to identify the proportion of each reference antibody contributing to the neutralization profile of the biological sample.

The methods also provide for identifying HCV epitope specificities for each neutralizing antibody.

The term “epitope” refers to a site on an antigen that elicits an immunological response in the subject to which it is administered and to which an immunoglobulin or antibody specifically binds. Often, an epitope will bind to an antibody generated in response to such sequence. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids, and not more than about 500 amino acids (or any integer therebetween), often contiguous amino acids, in a unique spatial conformation. There is no critical upper limit to the length of the fragment, which may comprise nearly the full-length of a protein sequence, or even a chimeric protein comprising two or more epitopes from the HCV polyprotein. An epitope herein is not limited to a polypeptide having the exact sequence of the portion of the parent protein from which it is derived. Indeed, viral genomes are in a state of constant flux and contain several variable domains that exhibit relatively high degrees of variability between isolates. Thus the term “epitope” encompasses sequences identical to the native sequence, as well as modifications to the native sequence, such as deletions, additions and substitutions (generally conservative in nature) that specifically bind an antibody that specifically binds the native sequence.

The methods described herein further provide that the neutralization profiles identify individual antibodies which bind to distinct HCV epitopes or are cross-reactive to related HCV epitopes. Moreover, the methods also including isolating the HCV neutralizing antibodies. For example, the method may include comprises culturing the host cell and isolating from the culture an antibody binding the HCV epitope. The method can further comprise screening the antibody in a cell culture system or in vivo to determine that it is a neutralizing antibody.

The methods described herein are also suitable for scaling, including, for example as a high throughput method.

### Vaccine

The methods herein also provide for a vaccine including a polypeptide having an Hepatitis C virus (HCV) epitope which induces an HCV neutralizing antibody, said antibody identified by the methods described herein.

The term “vaccine” as used herein is an antigenic preparation used to establish immunity to a disease or illness and thereby protect or cure the body from a specific disease or illness. Vaccines are either prophylactic and prevent disease or therapeutic and treat disease. Vaccines may contain more than one type of antigen. The term “vaccination” refers to the introduction of a vaccine into the body of a subject for the purpose of inducing immunity.

In examples, the vaccine includes an adjuvant. The term “adjuvant” refers a pharmacological or immunological agent that modifies the effect of other agents, such as a drug or vaccine. They are often included in vaccines to enhance the recipient's immune response to a supplied antigen, while keeping the injected foreign material to a minimum. Examples of adjuvants include alum, AIPO₄, aluminum hydroxide, alhydrogel, and Lipid-A and derivatives or variants thereof, Freund's incomplete adjuvant, Freund's complete adjuvant, liposomes, non-ionic block copolymers, and MF59C.1. MF59C.1 is a submicron oil-in-water emulsion, comprising squalene, sorbitan trioleate, and polysorbate80.

Also provided herein is an isolated hybrid cell producing an Hepatitis C virus (HCV) neutralizing monoclonal antibody identified by the methods described herein.

### Methods of Treating

Provided herein are methods for treating a subject infected with an Hepatitis C virus (HCV). The methods include administering to the subject a therapeutically effective amount of HCV neutralizing antibodies identified by the methods described herein in. In other examples, the methods of treating include administering the vaccines described herein.

For example, the terms “treat”, “treating” and “treatment” mean implementation of therapy with the intention of reduction in severity or frequency of symptoms, elimination of symptoms or their underlying cause, prevention of the occurrence of symptoms or their underlying cause, or improvement or remediation of damage. Moreover, the methods of treating include, for example, prophylactic treatment or prophylaxis, against means prevention of the occurrence of symptoms of HCV infection or their underlying cause.

Also provided herein are methods of identifying virus-specific neutralizing antibodies comprising, obtaining a biological sample from a subject having been infected with a virus; measuring neutralization of a virus by antibodies specific for the virus in the biological sample; generating a neutralization profile of each biological sample; deconvoluting the virus-specific neutralizing antibodies by generating a reference antibody neutralization profile; correlating the reference antibody neutralization profile to the biological sample's neutralization profile; and, identifying the virus neutralizing antibodies.

In examples, the methods of identifying virus-specific neutralizing antibodies includes viruses of adenoviruses, arenaviruses, bunyaviruses, flaviviruses, filoviruses, herpesviruses, noroviruses, orthomyxoviruses, poxviruses, papilloma viruses, paramyxoviruses, reoviruses, rhabdoviruses, retroviruses, or togaviruses.

Included herein is a method of preventing or treating HCV in a subject in need thereof. In further embodiments, the method comprises administering to the subject an effective amount of a composition comprising HCV neutralizing antibodies identified by the methods herein, or the vaccine identified for the methods described herein.

In other embodiments, the methods for treating HCV comprise administering to a subject a composition comprising HCV neutralizing antibodies or a vaccine identified and/or produced according to the methods described herein, in combination with methods for controlling the outset of symptoms. In particular, the combination treatment can include administering readily known treatments.

The described composition can be administered as a pharmaceutically or physiologically acceptable preparation or composition containing a physiologically acceptable carrier, excipient, or diluent, and administered to the tissues of the recipient organism of interest, including humans and non-human animals.

The HCV neutralizing antibodies or a vaccine identified and/or produced according to the methods described herein can be prepared by re-suspending in a suitable liquid or solution such as sterile physiological saline or other physiologically acceptable injectable aqueous liquids. The amounts of the components to be used in such compositions can be routinely determined by those having skill in the art.

In examples, for injectable administration, the composition (e.g., a composition comprising HCV neutralizing antibodies or a vaccine identified and/or produced according to the methods described herein) is in sterile solution or suspension or can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

In embodiments, a therapeutically effective amount of the composition (e.g., a composition comprising HCV neutralizing antibodies or a vaccine identified and/or produced according to the methods described herein) in humans can be any therapeutically effective amount. In one embodiment, the composition (e.g., a composition comprising HCV neutralizing antibodies or a vaccine identified and/or produced according to the methods described herein) is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In an embodiment, the composition (e.g., a composition comprising HCV neutralizing antibodies or a vaccine identified and/or produced according to the methods described herein) is administered once a week, or once every two weeks, or once every 3 weeks or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks).

### Pharmaceutical Compositions and Formulations

The present invention provides pharmaceutical compositions comprising an effective amount of a composition (e.g., a composition comprising HCV neutralizing antibodies or a vaccine identified and/or produced according to the methods described herein) and at least one pharmaceutically acceptable excipient or carrier, wherein the effective amount is as described above in connection with the methods of the invention.

In one embodiment, the composition (e.g., a composition comprising HCV neutralizing antibodies or a vaccine identified and/or produced according to the methods described herein) is further combined with at least one additional therapeutic agent in a single dosage form. In one embodiment, the at least one additional therapeutic agent comprises an antiviral drug.

Non-limiting examples of anti-viral agents that may be used in combination with a composition comprising HCV neutralizing antibodies or a vaccine identified and/or produced according to the methods described herein include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating muscle diseases or disorders are described herein.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition comprises an injectable form.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value. All percentages and ratios used herein, unless otherwise indicated, are by weight.

### General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

In embodiments, an antibody described herein may be a polyclonal antisera or monoclonal antibody. The term antibody may include any of the various classes or sub-classes of immunoglobulin (e.g., IgG, IgA, IgM, IgD, or IgE derived from any animal, e.g., any of the animals conventionally used, e.g., sheep, rabbits, goats, or mice, or human), e.g., the antibody comprises a monoclonal antibody, e.g., an HCV monoclonal antibody.

An “isolated antibody,” as used herein, is intended to refer to an antibody that is substantially free of other antibodies having different antigenic specificities (e.g., an isolated antibody that specifically binds HCV and is substantially free of antibodies that specifically bind antigens other than HCV). Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

An “antibody fragment” comprises a portion of an intact antibody, preferably the antigen binding and/or the variable region of the intact antibody. Non-limiting examples of antibody fragments include Fab, Fab*, F(ab′)₂ and Fv fragments; diabodies; linear antibodies; single-chain antibody molecules and multispecific antibodies formed from antibody fragments.

The invention may further comprise a humanized antibody, wherein the antibody is from a non-human species, whose protein sequence has been modified to increase their similarity to antibody variants produced naturally in humans. Generally, a humanized antibody has one or more amino acid residues introduced into it from a source which is non-human. These non-human amino acid residues are referred to herein as “import” residues, which are typically taken from an “import” antibody domain, particularly a variable domain.

The term “recombinant human antibody,” as used herein, includes all human antibodies that are prepared, expressed, created or isolated by recombinant means, such as (a) antibodies isolated from animals (e.g., sheep, rabbits, goats, or mice) that are transgenic or transchromosomal for human immunoglobulin genes, (b) antibodies isolated from a host cell transformed to express the human antibody, (c) antibodies isolated from a recombinant, combinatorial human antibody library, and (d) antibodies prepared, expressed, created or isolated by any other means that involve splicing of human immunoglobulin gene sequences to other DNA sequences.

An “antibody fragment” comprises a portion of an intact antibody, preferably the antigen binding and/or the variable region of the intact antibody. Examples of antibody fragments include Fab, Fab*, F(ab′)₂ and Fv fragments; diabodies; linear antibodies; single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. K and k light chains refer to the two major antibody light chain isotypes.

By the term “synthetic antibody” as used herein, is meant an antibody, which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned), synthesizing the RNA, or other technology, which is available and well known in the art.

The term “adjuvant” as used herein is defined as any molecule to enhance an antigen-specific adaptive immune response.

By “antigen” is defined as a molecule that provokes an immune response. This immune response may involve either antibody production, or the activation of specific immunologically-competent cells, or both. For example, any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used hereinThe term “disease” refers to any deviation from the normal health of a mammal and includes a state when disease symptoms are present, as well as conditions in which a deviation (e.g., hepatitis C virus (HCV)) has occurred, but symptoms are not yet manifested.

The term “epitope” as used herein refers to a sequence of at least about 3 to 5, at least about 5 to 10 or 15, and not more than about 1,000 amino acids (or any integer value between 3 and 1,000), which define a sequence that by itself or as part of a larger sequence, binds to an antibody generated in response to such sequence. There is no critical upper limit to the length of the fragment, which may comprise nearly the full-length of the protein sequence, or even a fusion protein comprising two or more epitopes from the HCV polyprotein. An epitope for use in the subject invention is not limited to a polypeptide having the exact sequence of the portion of the parent protein from which it is derived. Indeed, viral genomes are in a state of constant flux and contain several variable domains which exhibit relatively high degrees of variability between isolates. Thus the term “epitope” encompasses sequences identical to the native sequence, as well as modifications to the native sequence, such as deletions, additions and substitutions (generally conservative in nature).

“Immune response,” as the term is used herein, means a process involving the activation and/or induction of an effector function in, by way of non-limiting examples, a T cell, B cell, natural killer (NK) cell, and/or an antigen-presenting cell (APC). Thus, an immune response, as would be understood by the skilled artisan, includes, but is not limited to, any detectable antigen-specific activation and/or induction of a helper T cell or cytotoxic T cell activity or response, production of antibodies, antigen presenting cell activity or infiltration, macrophage activity or infiltration, neutrophil activity or infiltration, and the like.

An “immunological response” to an HCV antigen (including both polypeptide and polynucleotides encoding polypeptides that are expressed in vivo) or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the term “high-throughput detection” or “high-throughput screening” refers to a method for scientific experimentation especially used in drug discovery and relevant to the fields of biology and chemistry. Using robotics, data processing and control software, liquid handling devices, and sensitive detectors, high-throughput screening may allow a researcher to quickly conduct millions of chemical, genetic or pharmacological tests. Through this process one can rapidly identify active compounds, antibodies or genes which modulate a particular biomolecular pathway. The results of these experiments may provide starting points for drug design and for understanding the interaction or role of a particular biochemical process in biology (e.g., an HCV vaccine).

A “multi-well vessel”, as noted above, is an example of a substrate comprising more than one well in an array. Multi-well vessels useful in the invention can be of any of a variety of standard formats (e.g., plates having 2, 4, 6, 24, 96, 384, or 1536, etc., wells), but can also be in a non-standard format (e.g., plates having 3, 5, 7, etc., wells).

A “high throughput screen” or “HTS” as used herein refers to an assay which provides for multiple candidate agents, samples or test compound to be screened simultaneously. As further described below, examples of such assays may include the use of microtiter plates that are especially convenient because a large number of assays can be carried out simultaneously, using small amounts of reagents and samples. The methods are easily carried out in a multiwell format including, but not limited to, 96-well and 384-well formats and automated.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A “control” sample or value refers to a sample that serves as a reference, usually a known reference, for comparison to a test sample. For example, a test sample can be taken from a test subject, e.g., a subject with hepatitis C virus (HCV), and compared to samples from known conditions, e.g., a subject (or subjects) that does not have hepatitis C virus (HCV) (a negative or normal control), or a subject (or subjects) who does have hepatitis C virus (HCV) (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have hepatitis C virus (HCV) in a healthy or general population. The amount of a compound can be measured in a test sample and compared to the “normal control” level, utilizing techniques such as reference limits, discrimination limits, or risk defining thresholds to define cutoff points and abnormal values (e.g., for hepatitis C virus (HCV) or a symptom thereof). The normal control level means the level of one or more compounds or combined compounds typically found in a subject known not suffering from hepatitis C virus (HCV). Such normal control levels and cutoff points may vary based on whether a compounds is used alone or in a formula combining with other compounds into an index. Alternatively, the normal control level can be a database of compounds patterns from previously tested subjects who did not develop hepatitis C virus (HCV) or a particular symptom thereof (e.g., in the event the hepatitis C virus (HCV) develops or a subject already having hepatitis C virus (HCV) is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1, −2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J Pept. Prot. Res. 14:177-185 (1979); Spatola et al., Life Sci. 38:1243-1249 (1986); Hann, J. Chem. Soc. Perkin Trans. I 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982))

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Deconvolution Method

Neutralization of a diverse panel of nineteen genotype 1a and 1b HCV pseudoparticles (HCVpp) were measured by twenty anti-HCV human mAbs (13, 28). The HCVpp panel was selected to maximize genetic diversity among functional genotype 1 HCV E1E2 genes, and it contains 94% of amino acid polymorphisms present at greater than 5% frequency in genotype 1 HCV isolates from GenBank (29, 30).

A concordance between neutralization breadth of mAbs measured using this diverse genotype 1 panel and neutralizing breadth of the same mAbs measured using 112 HCV strains from genotypes 1-6 (13, 16, 30) was measured. Each mAb exhibited a distinct ranking of relative neutralization potency across the HCVpp in the panel, also known as a neutralization fingerprint or neutralization profile. The neutralization profile of each mAb was unique and reproducible, and these neutralization profiles also could be used to cluster functionally-similar mAbs into groups (28). Subsequently, plasma samples from HCV-infected persons also exhibited distinct neutralization profiles across the HCVpp panel (18), indicating that these neutralization profiles could be used to deconvolute HCV-specific NAbs in polyclonal plasma.

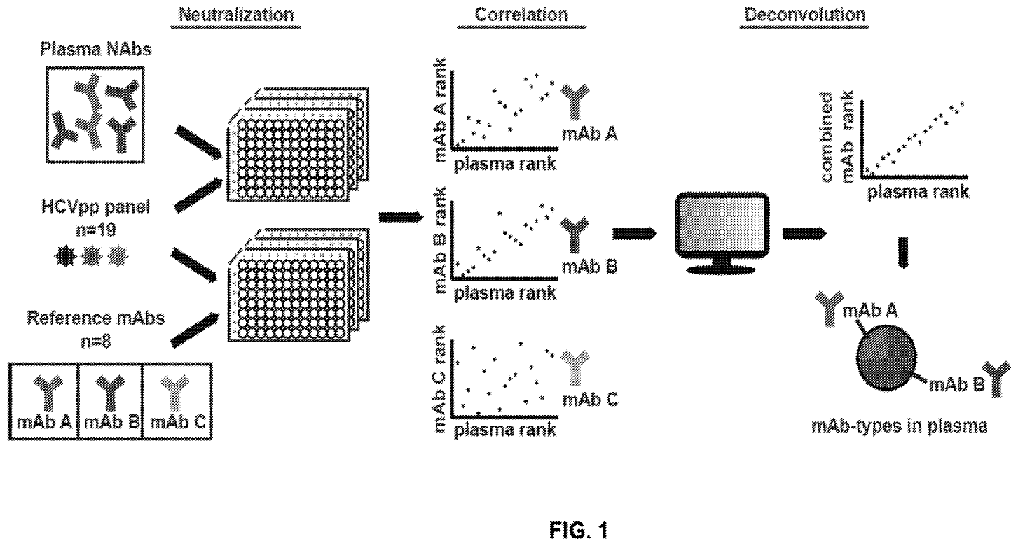

To deconvolute HCV-specific NAbs in plasma, neutralization was measured of the 19-HCVpp panel by plasma samples at a 1:100 or 1:20 dilutions, generating a neutralization profile for each sample (FIG. 1). To analyze these data, an algorithm developed by Georgiev, et al. was adapted for deconvolution of HIV-specific NAbs (31). The adapted algorithm correlated the reference antibody neutralization profiles with biological sample neutralization profiles, to identify the antibodies present in the biological sample. This algorithm adds reference mAb neutralization profiles in all possible proportions to generate an array of combined reference mAb neutralization profiles. The algorithm then identified the combined reference mAb neutralization profile with the strongest positive correlation with the plasma neutralization profile, thereby delineating the most likely proportion of each reference mAb contributing to the neutralization profile of the plasma sample.

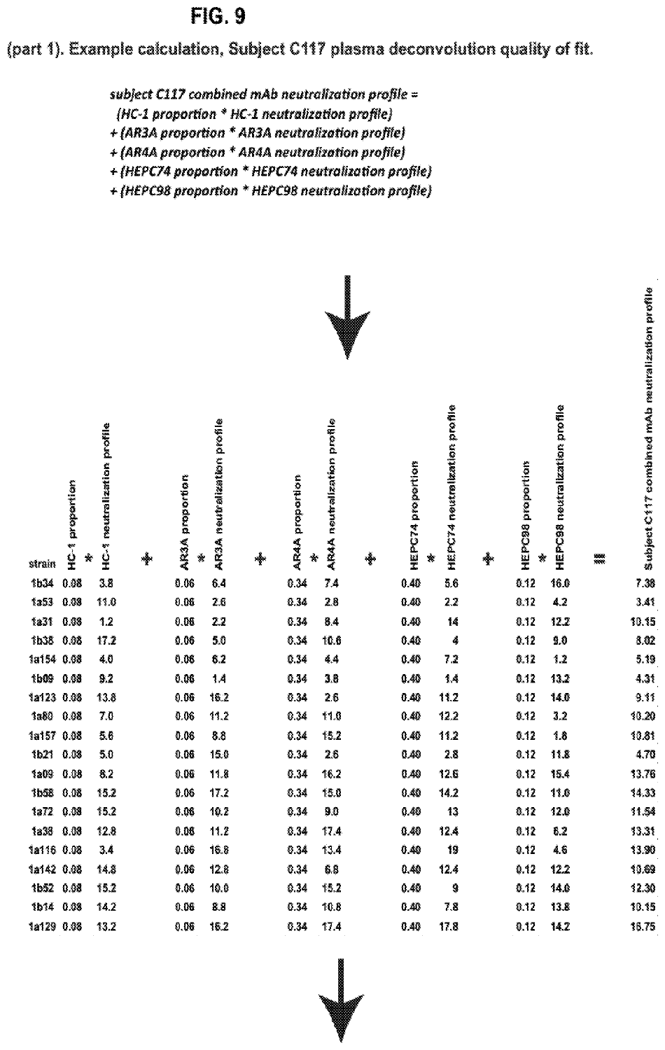

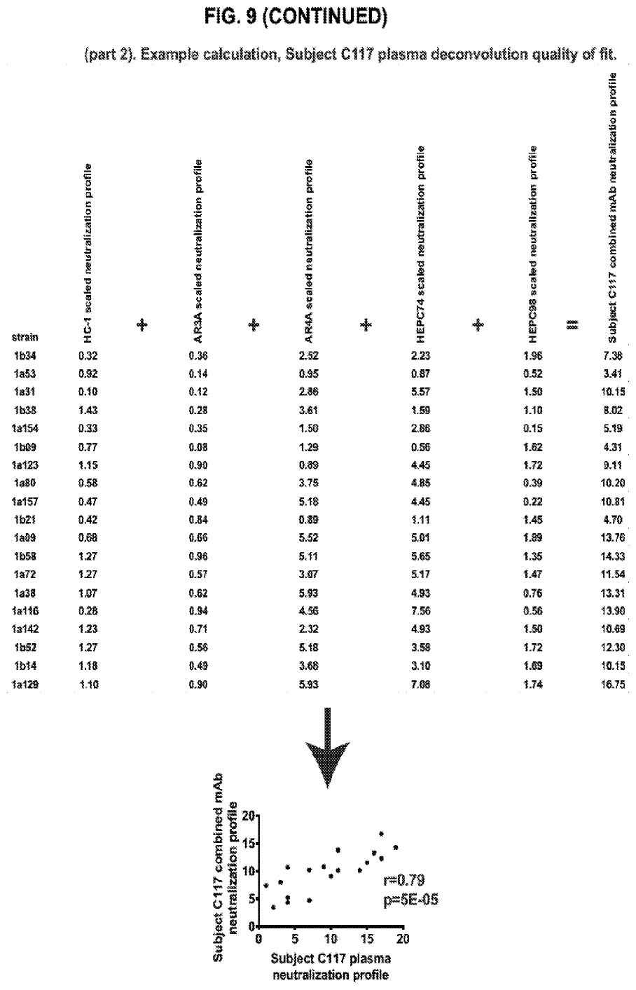

An additional step was added to the algorithm to quantitate quality of fit of each plasma deconvolution (See Materials and Methods, below and FIG. 9. For this quality analysis, reference mAb neutralization profiles were added in the proportions determined by the deconvolution, and then compared the resulting combined mAb neutralization profile to the corresponding plasma neutralization profile using Pearson correlation. A deconvolution was considered a good fit if the p value of this correlation was <0.05.

### Example 2: Selection of a Mab Reference Panel for Plasma Deconvolution

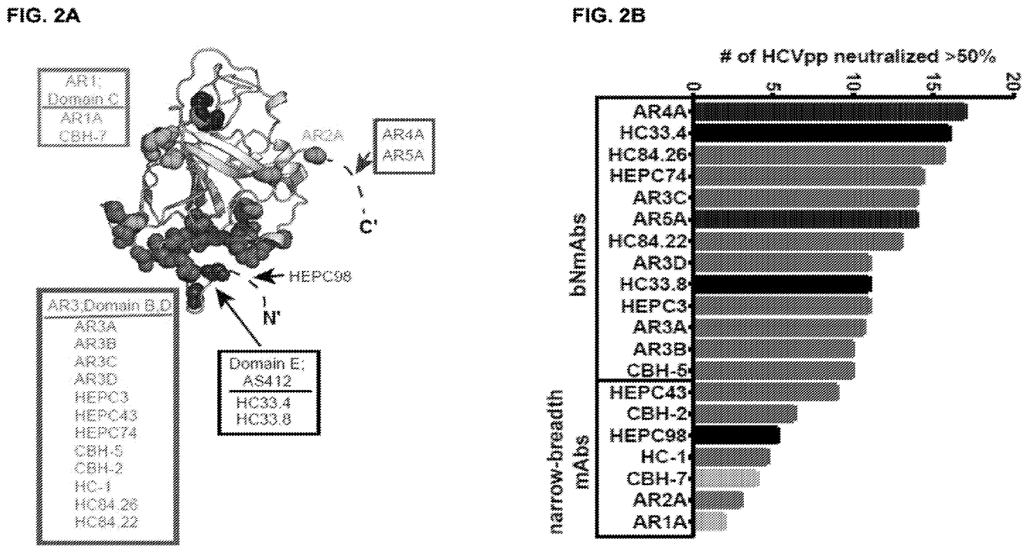

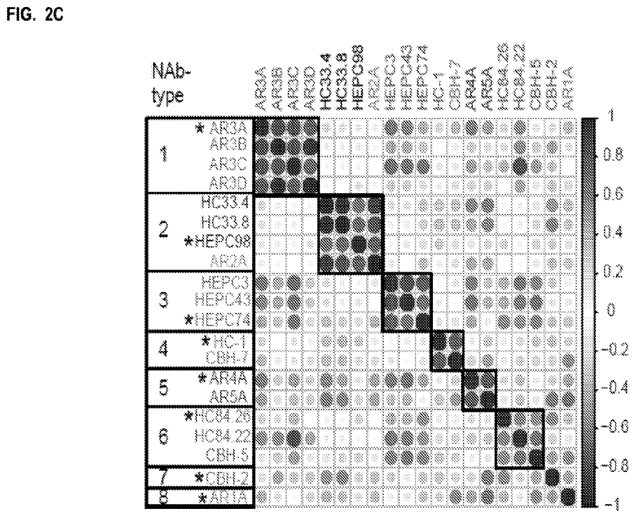

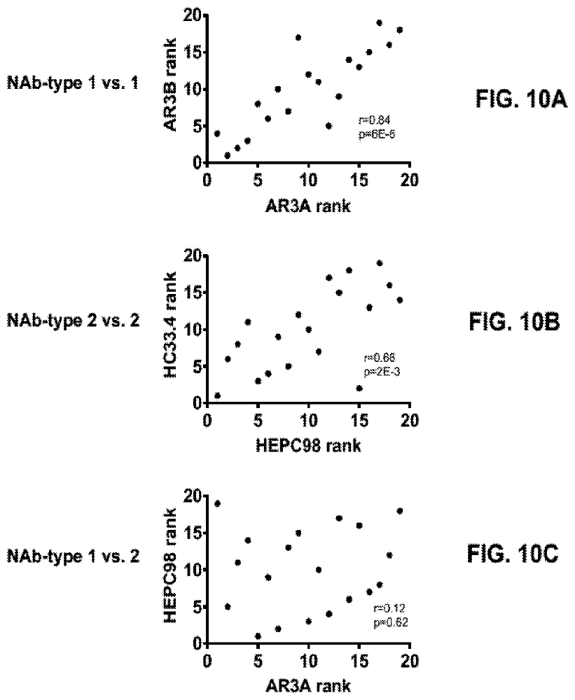

Many mAbs targeting distinct epitopes on HCV E1E2 have been isolated from the B cells of HCV-infected humans, but it is not known which of these mAbs might contribute to plasma neutralizing activity. To identify the most comprehensive, least redundant mAb reference panel for plasma deconvolution analysis, the previously tested set of 20 mAbs was analyzed. This set includes mAbs specific for a variety of epitopes on E1 and/or E2 145 (FIG. 2A) and it includes some of the most broadly-neutralizing mAbs described to date, as well as mAbs with more limited neutralizing breadth (FIG. 2B) (13, 19, 20, 26, 147 32, 33). To cluster this set of mAbs into NAb-types based on functional similarity, pairwise Pearson correlations between mAb neutralization profiles were calculated, and mAbs were grouped if the neutralization profile of each mAb in the group had a correlation (r)≥150 0.55 (p<0.02) with the profile of every other mAb in the group (FIGS. 2C and 10A-10C). At this r-value threshold, most mAbs with highly similar binding sites fell into the same group, and most mAbs with clearly distinct binding sites fell into distinct groups. For example, mAbs AR3A, AR3B, AR3C, and AR3D, which bind overlapping epitopes on E2 and compete for E2 binding (19, 34, 35), formed NAb-type 1. HC33.4, HC33.8, and HEPC98, which each bind near the N-terminus of E2, and also compete for E2 binding (13, 26, 27), formed NAb-type 2.

This analysis also revealed some functional relationships among mAbs that were not predicted by their binding sites (28). Some mAbs which are thought to bind to distinct antigenic sites, like HC-1 and CBH-7, appeared to be functionally related, forming a single NAb-type (Nab-type 4). Conversely, some mAbs binding to partially overlapping epitopes, like AR3A and HC-1 (36, 37), segregated into distinct NAb-types (NAb-type 1 and NAb-type 4, respectively). Some of these unexpected functional relationships are due to shared resistance mutations outside of the defined epitopes, which likely mediate indirect effects by allosteric means (28, 38-40). Overall, these data confirm that neutralization profiles can be used to identify individual mAbs or groups of mAbs with closely related binding epitopes.

Based on correlations between neutralization profiles, eighteen mAbs segregated into six NAb-types including two to four mAbs each, and two mAbs, CBH-2 and ARIA, did not group with other mAbs (FIG. 2C). To select the mAb reference panel for plasma deconvolution analysis, the most functionally distinct representative of each NAb-type was identified, which was the mAb from each group with the neutralization profile with the lowest correlations with the neutralization profiles of mAbs from other NAb-types. This analysis selected a reference panel comprising the following eight mAbs: AR3A, HEPC98, HEPC74, HC-1, AR4A, HC84.26, CBH-2, and ARIA. Notably, reference mAbs in this set exhibited a range of neutralizing breadth, from 3 of 19 HCVpp neutralized by 10 μg/mL of ARIA to 17 of 19 HCVpp neutralized by 10 μg/mL of AR4A.

For the purposes of this study, bNAbs were defined as NAbs capable of neutralizing ≥10 of 19 HCVpp by >50% when tested at 10 μg/mL concentration, and narrow-breadth NAbs as NAbs neutralizing <10 of 19 HCVpp under these same conditions. By these criteria, four of eight reference mAbs were bNAbs (AR4A, 182 HC84.26, HEPC74, and AR3A) and four of eight were narrow-breadth NAbs (ARIA, 183 HC-1, HEPC98, and CBH-2).

### Example 3: Deconvolution of Samples Containing Known mAbs

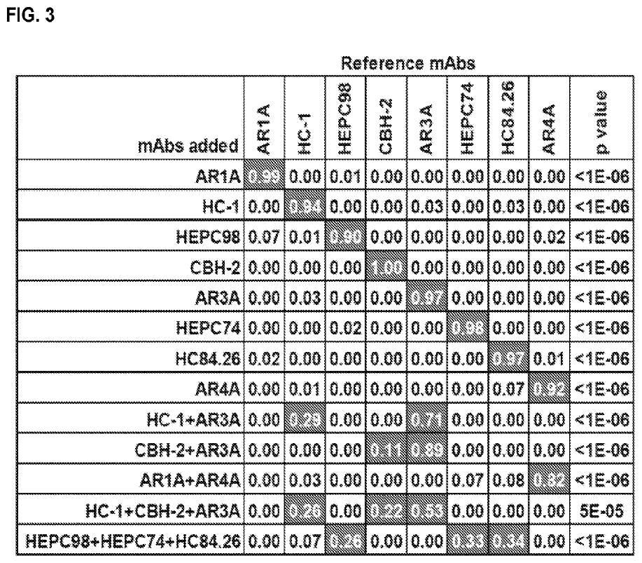

To validate the mAb reference panel and deconvolution method, neutralization testing was repeated against the 19-HCVpp panel with single reference mAbs or combinations of reference mAbs. These neutralization profiles were used as input into the deconvolution algorithm (FIG. 3). Deconvolution analysis correctly identified the mAb present in each single-mAb sample. Notably, these experiments confirmed that the deconvolution method can reliably discriminate between each of the eight reference mAbs, including AR3A, HEPC74, HC84.26, CBH-2, and HC-1, which target overlapping epitopes in the E2 front layer.

Samples containing combinations of two or three reference mAbs were also analyzed, with each reference mAb included in at least one combination. All mAbs except ARIA were identified correctly in the two-mAb combinations of HC-1+AR3A, CBH-2+AR3A, and AR1A+AR4A. Notably, proportions assigned to each mAb in the two mAb combinations were not equivalent, although the mAbs were combined at equivalent concentrations, likely reflecting relative potency of the mAbs.

Two different combinations of three mAbs were also tested. When HC-1, CBH-200 2, and AR3A were combined at equivalent concentrations, all three mAbs were identified in the deconvolution analysis at proportions of 0.26, 0.22, and 0.53, respectively. HEPC98, HEPC74, and HC84.26 also were identified correctly in combination, with proportions of 0.26, 0.33, and 0.34, respectively. Taken together, these data confirm that deconvolution analysis can identify individual mAbs alone or in polyclonal mixtures, although some mAbs, such as ARIA, may be more difficult to detect in a mixture with other mAbs.

Since low-level false-positive proportions were identified in some samples, a true-positive threshold proportion >0.1 was set, which resulted in 95% sensitivity and 100% specificity in FIG. 3 experiments. Proportions greater than 0.1 were considered positive in all subsequent analyses.

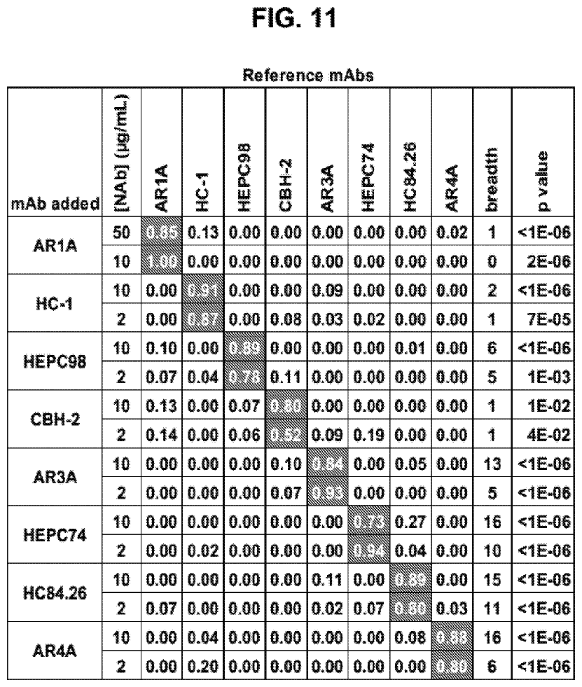

Because neutralization profiles are a relative ranking of neutralization of HCVpp by each NAb, rather than absolute neutralization values, deconvolution results would be consistent across multiple mAb concentrations. To test this hypothesis, deconvolution analysis was performed using neutralization profile results measured using two different concentrations of each reference mAb (FIG. 11). The deconvolution result for each mAb was consistent at either 10 or 2 μg/mL mAb concentrations (50 or 10 μg/mL for ARIA), confirming that neutralization profiles remain consistent even when percentage neutralization values are very low. Notably, narrow-breadth mAbs ARIA, HC-1, and CBH-2 were correctly identified based on relative neutralization of HCVpp across the panel even at concentrations of 2 to 10 μg/mL, when each mAb neutralized only 0 or 1 of 19 HCVpp in the panel by more than 50%, and 3 to 6 HCVpp by more than 25%.

### Example 4: Concordance Between Plasma NAB Deconvolution and Mabs Isolated from B Cells of the Same Subject

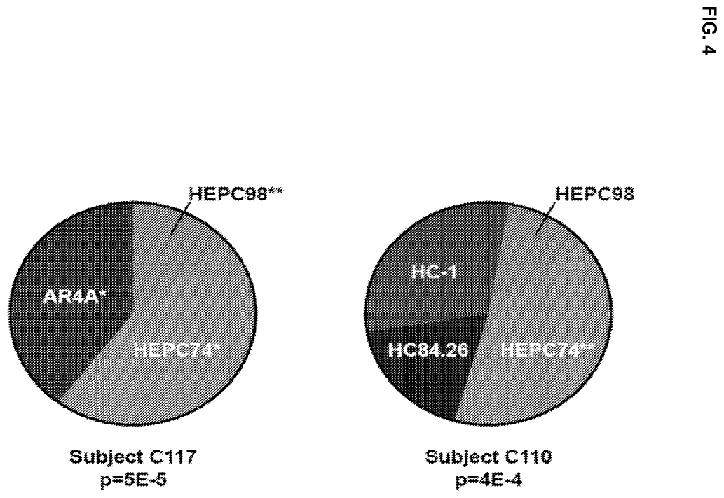

To validate the plasma deconvolution method by a second approach, deconvolution analysis was performed on plasma of two human donors, subjects C117 and C110, from previously isolated mAbs from peripheral blood B cells (FIG. 4). C117 was the donor of B cells producing reference mAb HEPC98 as well as mAbs designated HEPC3 and HEPC84, which are HEPC74-type or AR4A-type NAbs, respectively (FIGS. 2A-2C and (13)). Therefore, it was hypothesized that one should detect HEPC98-type, HEPC74-type, and AR4A-type NAbs in C117 plasma. C110 was the donor of B cells producing reference mAb HEPC74, so it was hypothesized that one should detect HEPC74-type NAbs in C110 plasma. For each subject, plasma obtained prior to HCV clearance was analyzed, using averaged neutralization profiles obtained using 1:20 and 1:100 dilutions of each plasma sample as input into the deconvolution analysis (see Materials and Methods, below). HEPC98-type, HEPC74-type, and AR4A-type NAbs were each identified in C117 plasma (proportions=0.12, 0.40, and 0.34, respectively), and HEPC74-type NAbs were identified in C110 plasma (proportion=0.36). Notably, HC-1-type, HC84.26-type, and HEPC98-type NAbs were also detected in C110 plasma (proportions=0.27, 0.16, and 0.10, respectively), although they were not isolated from his B cells, which may reflect false positive plasma deconvolution results or the relative inefficiency of mAb isolation. Taken together, these results show that mAbs isolated from the B cells of two subjects also were identified in the plasma of the same subjects by deconvolution analysis.

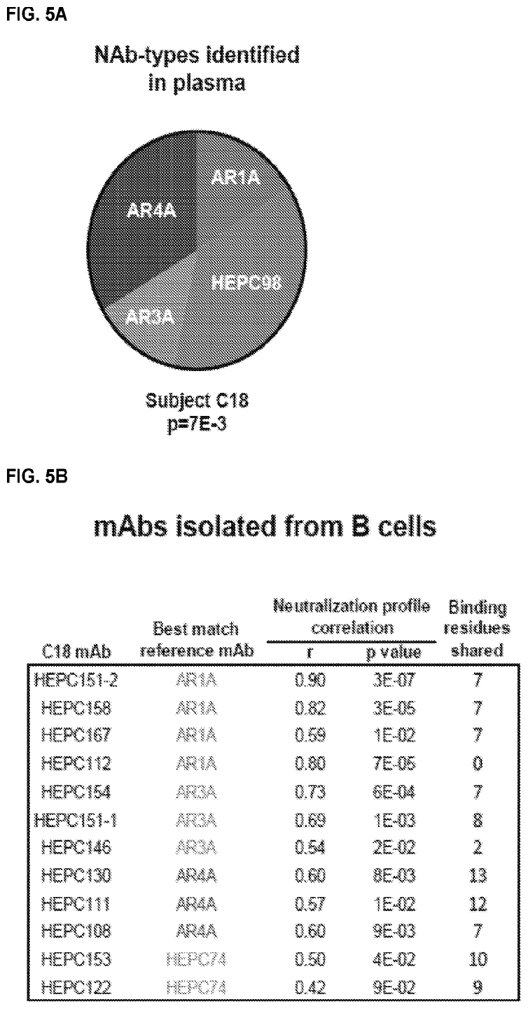

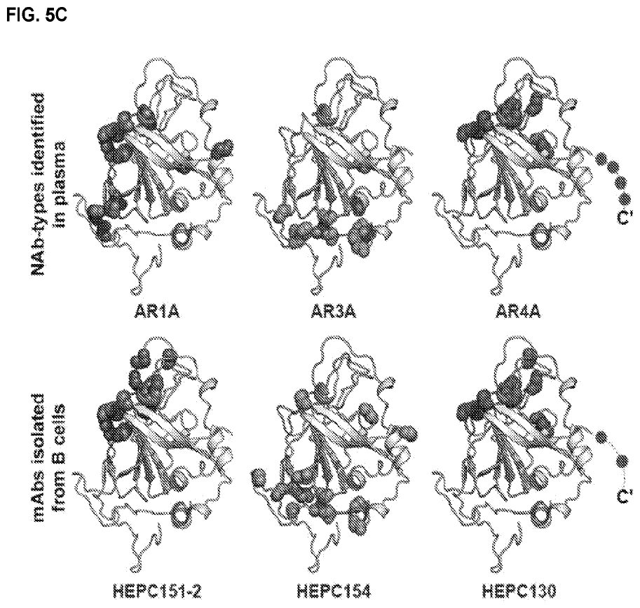

To further validate the deconvolution method, the plasma NAbs of a different HCV-positive subject were deconvoluted, designated C18, using an average of neutralization profiles obtained using 1:20 and 1:100 dilutions of plasma as input into the deconvolution analysis. mAbs were isolated from B cells of the same subject (FIGS. 5A-5C). Unlike C117 and C110, some of the neutralizing activity in C18 plasma was attributed to ARIA-type and AR3A-type NAbs (proportions 0.16 and 0.13, respectively), with the remainder of activity attributed to HEPC98-type and AR4A-type NAbs (proportions 0.37 256 and 0.33, respectively) (FIG. 5A).

Isolation and characterization of thirteen mAbs from B cells of this subject was described (16). In that study, twelve of thirteen mAbs displayed neutralizing activity across the HCVpp panel at 50 μg/mL concentration. Neutralization profiles were measured for these twelve mAbs, and mapped of up to fifteen (range 3 to 15) probable E1E2-binding residues for each C18 mAb and reference 261 mAb based on binding to a comprehensive alanine mutant library spanning the E1 and E2 262 proteins of strain H77. Here, the reference mAb (ARIA, HC-1, CBH-2, 263 HEPC98, AR3A, HEPC74, HC84.26, or AR4A) was identified that was most similar to each C18 mAb based on neutralization profile correlations and/or shared probable E1E2-binding residues (FIG. 5B).

Neutralization profiles of ten of twelve mAbs isolated from C18 B cells correlated significantly with profiles of reference mAb-types identified in plasma by the deconvolution analysis (ARIA, AR3A, or AR4A) which was significantly greater than the concordance between C18 plasma NAbs and C18 B cell mAbs expected by chance (83% vs. 44%, p=0.007 by binomial test, see Materials and Methods, below). Nine of twelve C18 mAbs (75%) shared both neutralization profiles and E1E2-binding residues with reference mAb-types identified in plasma by the deconvolution analysis. In addition, 24 E1E2-binding residues of C18 B cell mAbs were 7.5-fold more likely to fall at binding residues of reference mAb-types identified in plasma by deconvolution analysis than at other residues in E1 or E2 (p<1E-15 by Fisher's exact test).

Three of twelve C18 B cell mAbs (25%) were not predicted by the plasma deconvolution analysis. Two C18 mAbs matched best with reference mAb HEPC74, which was not detected in plasma. In addition, C18 mAb HEPC112 did not share E1E2-binding residues with any reference mAb, although the HEPC112 neutralization profile correlated strongly with reference mAb ARIA. This correlation between neutralization profiles of ARIA and HEPC112 is explained in part by the strong bias of each mAb for neutralization of genotype 1a strains over 1b strains, indicating that this method may falsely attribute some genotype 1a-biased plasma NAb responses to ARIA-type NAbs. HEPC98-type Nabs were identified in plasma but not isolated from B cells, which may indicate a false positive plasma deconvolution result or the relative inefficiency of mAb isolation.

Overall, nine of twelve C18 mAbs isolated from B cells (75%) matched plasma deconvolution-identified reference mAbs by both neutralization profiles and binding residues. FIG. 5C shows the location on the E2 structure of probable binding residues of three reference mAb-types identified in C18 plasma by the deconvolution analysis (ARIA, AR3A, AR4A) and three C18 mAbs with which each had a positively correlated neutralization profile and shared binding residues (HEPC151-2, HEPC154, HEPC130). Taken together, these data indicate that the NAb-types detected in plasma by the deconvolution analysis were strongly predictive of both the functional phenotypes and E1E2-binding residues of mAbs subsequently isolated from B cells of the same subject.

### Example 5: Deconvolution of Nab-Types in Plasma of Humans with Subsequent Clearance or 300 Persistence of HCV Infection

For a prior study, HCVpp panel was used to measure neutralizing breadth of plasma samples isolated from twenty-one subjects with subsequent clearance of HCV infection, and forty-two subjects with subsequent persistence of infection (11). Age, sex, race, follow-up interval, HCV infection genotype and interferon lambda 3-related rs12979860 305 allele frequencies were similar between these Persistence and Clearance groups, as previously described (11). Plasma was isolated from Clearance subjects at the last viremic time point prior to clearance of infection, and plasma from Persistence subjects was time-matched with the Clearance plasma samples for duration of infection. That study demonstrated that acute-infection plasma from Clearance subjects was significantly more broadly-neutralizing than plasma from Persistence subjects. The deconvolution method was applied to identify the NAbs responsible for this plasma neutralizing breadth.

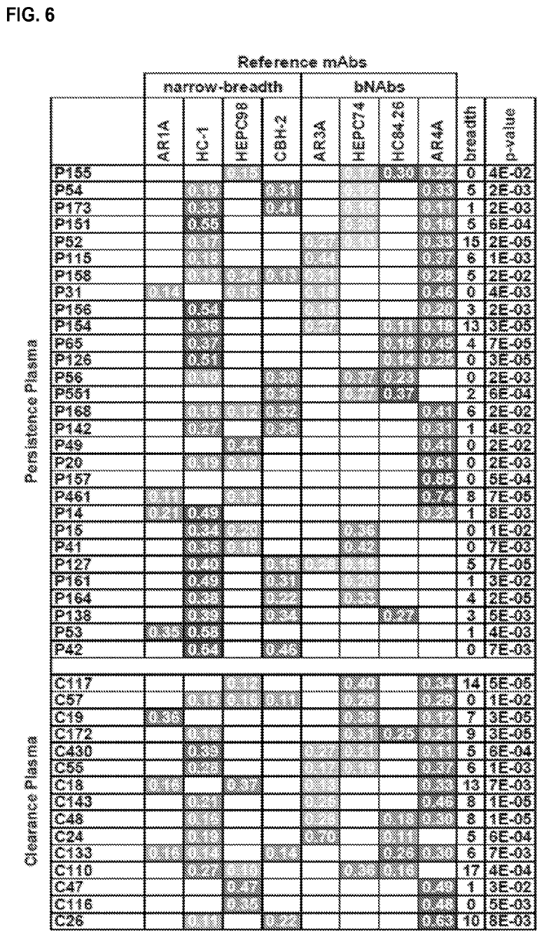

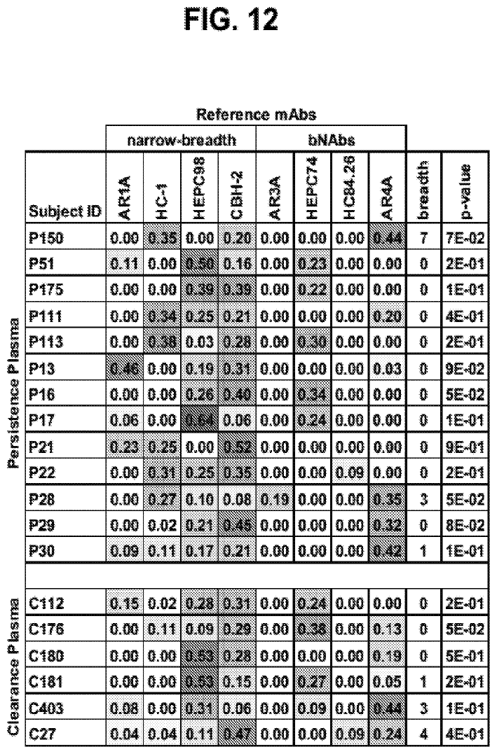

Neutralization profiles of these sixty-three plasma samples were averaged from neutralization results measured in independent experiments at 1:20 and 1:100 plasma dilutions, and used these neutralization profiles to deconvolute NAb-types present in the samples (FIG. 6). Forty-four (70%) of the samples (persistence n=29, clearance 318 n=15) had a good fit in the deconvolution analysis (p<0.05), suggesting that the neutralizing activity of these samples could be attributed to one or more of the mAb-types in the reference panel. The proportion of subjects with good fit in the deconvolution analysis did not differ significantly between the Persistence and Clearance groups (69% 322 vs. 71%, p=1, Fisher's exact test). Results for samples with poor fit in the deconvolution analysis are shown in FIG. 12. Although the NAbs present in these samples were not identified with confidence, the samples with poor fit displayed very low neutralizing breadth (mean breadth=1 of 19 HCVpp neutralized), indicating that the poor fit could more likely be explained by absent or very low titer bNAbs in these samples, rather than the presence of novel bNAbs not represented by the reference mAb panel.

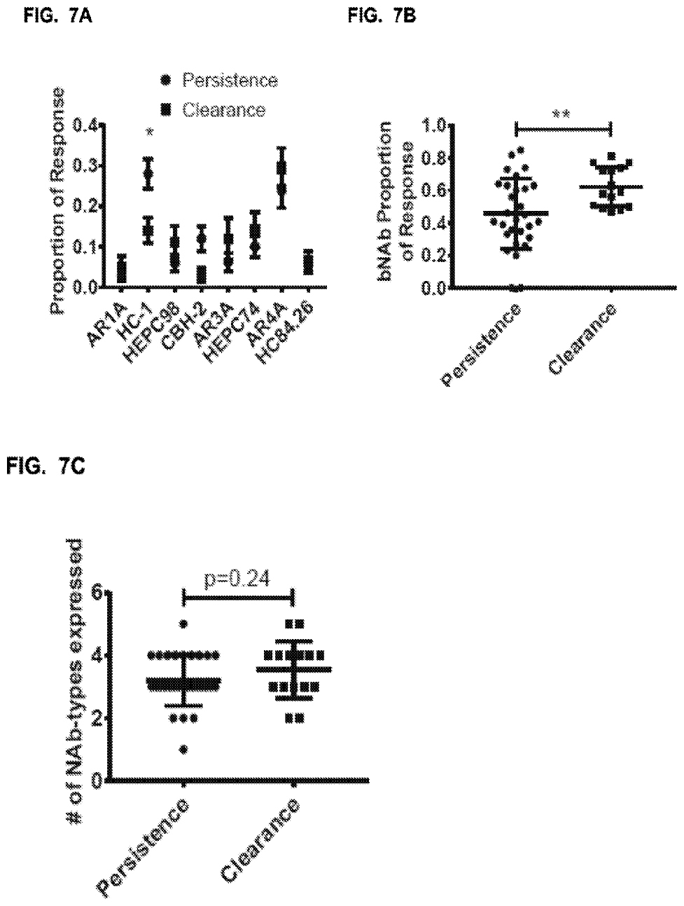

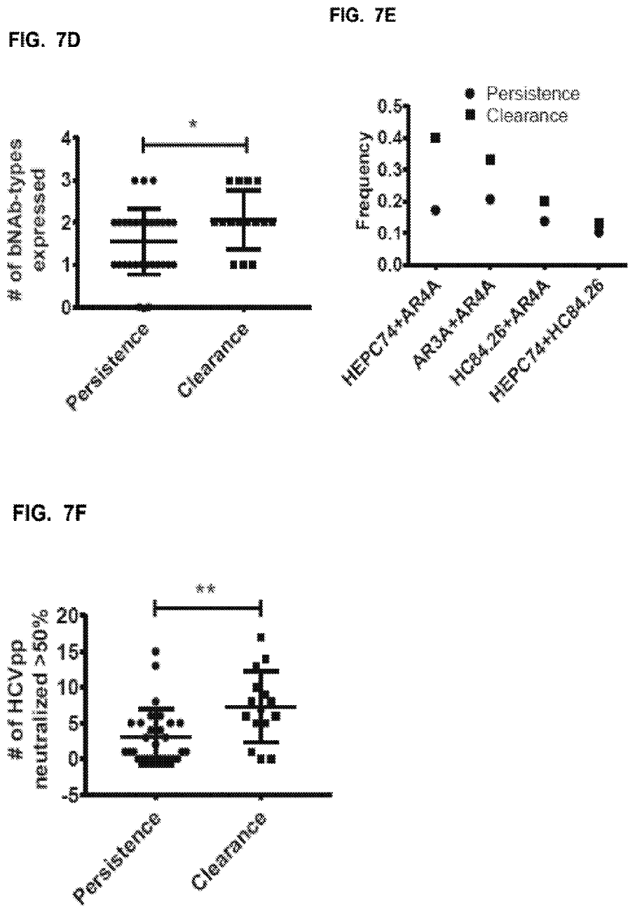

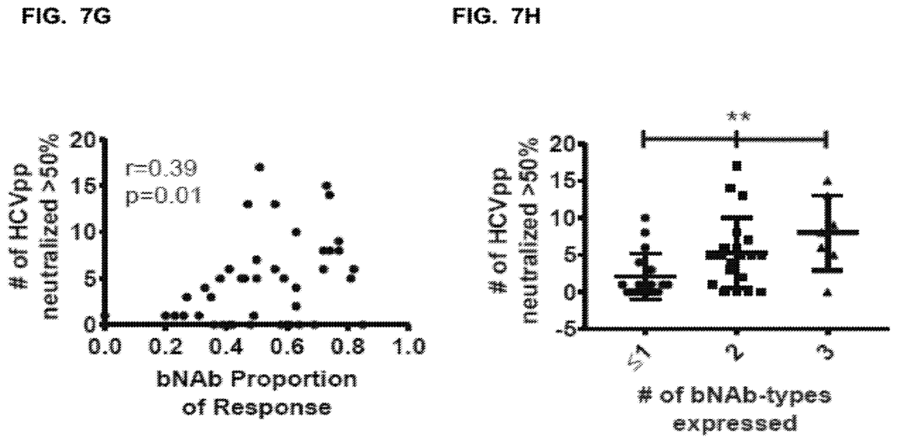

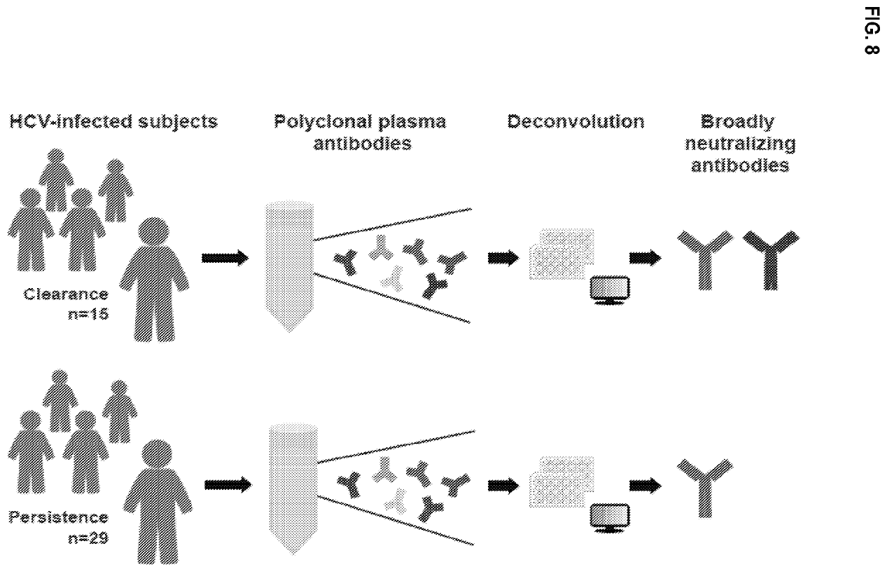

All subsequent analyses were performed using only data from the plasma samples with good fit in the deconvolution analysis (FIGS. 7A-7H). A significantly higher proportion of the antibody response was devoted to narrow-breadth HC-1-type NAbs in Persistence subjects relative to Clearance subjects (mean Persistence vs. Clearance proportions 0.28 333 vs. 0.14, p=0.03 by one-way ANOVA adjusted for multiple comparisons) (FIG. 7A). The mean proportion of the antibody response devoted to each bNAb-type (i.e., AR3A, 335 HEPC74, HC84.26, or AR4A) was similar for Persistence and Clearance subjects, although there was a trend toward a higher proportion for each bNAb-type in Clearance relative to Persistence subjects. Taken together, these data indicate that focusing of the immune response toward narrow-breadth HC-1-type NAbs was associated with persistence of HCV infection, and no single NAb-type was strongly associated with HCV clearance.

Given prior data indicating that some bNAbs in combination are synergistic with enhanced neutralizing breadth (16, 41, 42), it was hypothesized that combinations of bNAbs, rather than individual responses, might be associated with HCV clearance. Therefore, the total proportion of the neutralizing response devoted was compared to bNAbs (i.e., sum of AR4A, HC84.26, HEPC74, and AR3A proportions) in Clearance or Persistence subjects (FIG. 7B). Remarkably, significantly higher total bNAb proportions were measured in Clearance vs. Persistence subjects (mean proportion 0.62 vs. 0.46, p=0.009 by two-tailed T test). Although the total number of reference NAb-350 types detected per plasma sample did not differ between Clearance and Persistence groups (mean=3.5 vs 3.2 NAb-types, p=0.24 by two-tailed T test) (FIG. 7C), a significantly higher number of distinct bNAb-types per plasma sample were detected in Clearance vs. Persistence subjects (mean # of bNAb-types per sample 2.1 vs. 1.6, p=0.04 354 by two-tailed T test) (FIG. 7D). The most common 2-bNAb combinations detected in Clearance plasma were HEPC74+AR4A (6 of 15 subjects), AR3A+AR4A (5 of 15 subjects), and HC84.26+AR4A (3 of 15 subjects) (FIG. 7E). These data indicate that focusing of the humoral response toward bNAbs rather than narrow-breadth NAbs, and simultaneous expression of multiple bNAb-types in a single plasma sample was associated with HCV clearance.

### Example 6: Focusing of the Humoral Response Toward bNAbs and Expression of Multiple bNAb-362 Types was Associated with Greater Plasma Neutralizing Breadth

To investigate the mechanism by which focusing of the humoral response toward bNAbs, and expression of multiple bNAb-types was associated with clearance of infection, the association between these parameters and plasma neutralizing breadth was measured. First, neutralizing breadth of Persistence and Clearance plasma samples were compared, limiting the analysis to samples with good fit in the deconvolution analysis (FIG. 7F). A significantly greater neutralizing breadth by the Clearance samples (mean # of HCVpp neutralized by 1:100 dilution of Clearance vs. Persistence plasma 7.3 vs. 3.1, 370 p=0.003 by two-tailed T test), was observed which was consistent with the prior analysis of the full set of 63 plasma samples (11).

Next, the correlation between the total bNAb proportion of each plasma sample (i.e., sum of AR4A, HC84.26, HEPC74, and AR3A proportions) was measured and the neutralizing breadth of that sample (FIG. 7G). A statistically significant positive correlation between these variables (r=0.39, p=0.01 by Pearson's method) was observed. Notably, a significant correlation between the number of bNAb-types expressed in each plasma sample and the neutralizing breadth of that sample (FIG. 7H) was observed. The mean neutralizing breadth of plasma samples expressing 1 or fewer bNAb-types, 2 bNAb-types, or 3 bNAb-types was 2.1, 5.3, or 8 HCVpp neutralized, respectively (p=0.004 for the trend by one-way ANOVA).

Taken together, these data indicate that focusing of the humoral response toward bNAbs rather than narrow-breadth NAbs, and simultaneous expression of multiple bNAb-types was associated with greater plasma neutralizing breadth as well as clearance of HCV infection.

### DISCUSSION

HCV vaccine development has been hampered by incomplete understanding of correlates of protective immunity in humans, and by inadequate methods for assessing antibody responses induced by candidate vaccines. These deficits were addressed through the development of an assay that deconvolutes the anti-HCV NAbs in polyclonal plasma. Unexpectedly, it was found that most human subjects with subsequent clearance or persistence of HCV infection developed at least one bNAb during acute infection. Importantly, focusing of the humoral response toward bNAbs rather than narrow-breadth NAbs, and simultaneous expression of multiple bNAb-types was associated with greater plasma neutralizing breadth and with HCV clearance.

These results support prior work indicating that HCV vaccines should induce multiple bNAb-types (42-45), and these findings were extended by demonstrating that it may be important to induce HEPC74, AR3A, or HC84.26-like bNAbs in combination with AR4A-like bNAbs, while avoiding induction of more narrow-breadth NAbs like HC-1. Broadly-neutralizing AR3A, HEPC74, and HC84.26 as well as relatively narrow-breadth HC-1 and CBH-2 bind to overlapping conformational epitopes, which may complicate efforts to induce one NAb-type without the other. It is possible that this complication has played a role in the relatively limited neutralizing breadth induced by candidate vaccines to date (46, 47), if HC-1 type NAbs were induced at the expense of more broadly-neutralizing E2 front layer-specific antibodies. Targeting of more variable epitopes at the expense of more conserved, broadly neutralizing epitopes, commonly known as “deceptive imprinting”, has been described previously for HIV-1 and influenza (48).

Alternatively, HC-1-type NAbs may represent an early response that could mature to a HEPC74 or AR3A-like response with appropriate T cell help. Further longitudinal studies of vaccines or human subjects with persistent infection will be necessary to answer this question. Notably, traditional methods used to map epitopes of vaccine-induced NAbs, such as E1E2 binding competition between serum and reference mAbs, would likely not discriminate between the desired E2 front-layer specific bNAbs and less desirable narrow-breadth HC-1-type NAbs targeting the same antigenic site. This deconvolution method also provides a considerable advantage relative to post-vaccination neutralizing breadth testing alone, since breadth might result from targeting of one or multiple epitopes. In contrast, deconvolution analysis identifies the epitopes targeted, which could facilitate rational antigen design and iterative vaccine improvement to favor induction of bNAb combinations associated with clearance of natural infection. Fortunately, this method is high throughput and requires only about 100 μl of plasma, so it would be ideal for testing of longitudinal plasma samples or for optimizing vaccine protocols when only small plasma volumes can be obtained.

Further standardization of the assay to quantitate actual concentrations of individual NAb-types in plasma include testing of multiple reference mAb combinations with each mAb at multiple different concentrations. The assay does not detect non-neutralizing antibodies, which may also play a role in controlling infection (49), or in shaping the NAb response by competing with NAbs for overlapping epitopes on E1 and E2 (22-27). The NAbs that were detected infrequently in this study may be rare in plasma, or they may be present but effectively out-competed for E2 binding by non-neutralizing antibodies. Further work is needed to understand this relationship between neutralizing and non-neutralizing antibodies against HCV. Finally, this method does not measure plasma neutralizing titers, which may play an important role in the outcome of infection.

In summary, a method that deconvolutes anti-HCV NAbs in polyclonal plasma was developed, and this method was used to identify NAb-types in acute infection plasma of human subjects with subsequent clearance or persistence of HCV. No single Nab-type was strongly associated with HCV clearance. Remarkably, however, focusing of the humoral response toward bNAbs rather than narrow-breadth NAbs, and simultaneous expression of multiple bNAb-types was associated with greater plasma neutralizing breadth and with HCV clearance. These studies inform new strategies for vaccine development by identifying bNAb combinations in plasma associated with clearance of HCV, while also providing a high-throughput assay that could identify these responses after vaccination trials.

### Materials and Methods

### Study Subjects

Plasma samples and PBMC were obtained from subjects in the BBAASH cohort (10).

Source of mAbs

MAbs CBH-2, CBH-7, HC-1, CBH-5 (50), HC84.22, HC84.26 (20), HC33.4, HC33.8 (26) were a kind gift of Dr. Steven Foung (Stanford University School of Medicine, Palo Alto, Calif.). MAbs ARIA, AR2A, AR3A, AR3B, AR3C, AR3D 467 (19), AR4A, and AR5A (32) were a kind gift of Dr. Mansun Law (Scripps Research Institute, La Jolla, Calif.). All other antibodies were isolated in the laboratory of James E. Crowe, Jr. (13, 16).

### Cell Lines

HEK293T cells and Hep3B cells were obtained from ATCC.

### HCVpp Neutralization Assay

HCVpp were produced by Lipofectamine-mediated transfection of HCV E1E2, pNL4-3.Luc.R-E-, and pAdVantage (Promega) plasmids into HEK293T cells as previously described (28, 51, 52). Neutralization assays were performed as described previously (53, 54). mAbs at 10 or 50 μg/mL, or heat-inactivated plasma samples at 1:100 or 1:20 dilution were incubated with HCVpp for one hour at 478 37° C. prior to addition to Hep3B cells in duplicate. Medium was changed after 5 hours, and cells were incubated for 72 hours prior to measurement of luciferase activity in cell lysates in relative light units (RLU). Only HCVpp preparations producing at least 1E6 RLU were used for neutralization experiments, and HCVpp input was normalized to 1 to 6E6 RLU. Nonspecific human IgG (Sigma-Aldrich) at 50 μg/mL was used as a negative control. Fraction unaffected (Fᵤ) was calculated by the formula: Fᵤ=RLU_{mAb} or RLUₚₗₐₛₘₐ/RLU_{nonspecific} IgG. Fᵤ values were converted to a neutralization profile for each mAb or plasma sample (rank order of HCVpp neutralization with the most sensitive HCVpp ranked 1 and the least sensitive HCVpp ranked 19) for input into the deconvolution algorithm.

### Deconvolution of NAb Specificities in Plasma

Neutralization of a diverse panel of 19 genotype 1 HCVpp (strains 1a09, 1a31, 1a38, 1a53, 1a72, 1a80, 1a116, 1a123, 1a129, 1a142, 1a154, 1a157, 1b09, 1b14, 1b21, 1b34, 1b38, 1b52, 1b58) by 20 HCV-specific mAbs at 10 μg/mL concentration was quantitated for prior studies, generating a neutralization profile for each mAb, as previously described (13, 28). Once the final panel of 8 reference mAbs was selected, neutralization profiles were measured for each of these mAbs in 4 additional independent experiments. Neutralization profiles for each reference mAb were averaged across all five experiments by averaging the rank value for each HCVpp across all experiments, generating a final neutralization profile for each reference mAb (Table 1). Plasma samples from subjects with clearance (n=21) or persistence (n=42) of HCV infection were previously tested at a 1:100 dilution for neutralization of the same 19 HCVpp panel (11), except that HCVpp 1b21 and 1a116 were replaced by closely related HCVpp 1b20 (differing from 1b21 by 1 aa) and 1a114 (differing from 1a116 by 10 aa), due to greater availability of these HCVpp stocks at the time of testing.

Because reference mAbs were not tested against 1b20 and 1a114, plasma neutralization results for these HCVpp were not included in neutralization profiles used for deconvolution analysis. The same plasma samples were tested in a second independent experiment at a 1:20 dilution for neutralization of the same 19-HCVpp panel used to determine reference mAb neutralization profiles, including strains 1a116 and 1b21. Neutralization data for eight HCVpp (1b34, 1a53, 1b38, 1a123, 1a80, 1b58, 1a142, 509 and 1b14) were discarded from this second experiment due to inadequate HCVpp infectivity (<1E6 RLU). Neutralization profiles were averaged across these two independent experiments to generate a final neutralization profile for each plasma sample (Table 2). Deconvolution analysis was performed using code developed by Georgiev, et al. (31) in Wolfram Mathematica, v. 11.0 to delineate the relative proportion of each reference mAb present in each plasma sample. Rank order of plasma neutralization profiles were reversed prior to analysis to fit input requirements of the deconvolution program. Calculating fit of plasma deconvolution (See example calculation, FIG. 9)

For each plasma sample deconvolution, a scaled neutralization profile was calculated for each reference mAb by multiplying the neutralization profile of that reference mAb by the proportion of that mAb-type (range 0-1) calculated to be present in the plasma sample. These eight scaled reference mAb neutralization profiles then were added to generate a combined reference mAb neutralization profile. This combined reference mAb neutralization profile was compared to the actual plasma neutralization profile by Pearson correlation. NAb deconvolution for any plasma sample was considered a good fit if the correlation between the combined mAb neutralization profile and the plasma neutralization profile had p<0.05 by two-sided test.

### Shotgun Mutagenesis Epitope Mapping

Epitope mapping of mAbs was previously described (16, 34). Briefly, comprehensive alanine scanning mutagenesis of an expression construct for HCV E1/E2 (genotype 1a, strain H77) changed each residue to alanine (with alanine residues changed to serine) to create a library of clones, each representing an individual point mutant, covering 552 of 555 target E1/E2 residues. Binding of each mAb to wild type E1E2 and to each of these alanine mutants was quantitated to identify up to 15 residues where mutation to alanine mediated the greatest reduction in mAb binding, and also reduced binding by at least 50% relative to binding to wild type H77 E1E2. Probable binding residues of reference mAbs were identified through re-analysis of primary data included in prior publications (13, 36, 37, 50).

### Statistical Analysis

Statistical analyses were performed in Prism (GraphPad software, v7.02). Probability of concordance between neutralization profiles of NAb-types identified in C18 plasma and mAbs isolated from B cells arising by chance was calculated using a binomial test and the null hypothesis that each mAb isolated would fall by chance in one of nine groups, either matching one of the eight reference mAbs, or none of them. Significance of increased likelihood of C18 mAb binding residues falling at binding residues of deconvolution-identified reference mAbs was calculated using Fisher's exact test and the following data: 8 to 15 binding residues were identified for each of 12 C18 mAbs; 3 to 15 binding residues were known for each of 4 mAbs identified in plasma; and there were 552 possible E1E2-binding residues for each mAb.

### Study Approval

The protocol was approved by the Institutional Review Board of the Johns Hopkins Hospital, and informed consent was obtained from all study participants.

TABLE 1
Neutralization profiles of reference NAbs. The neutralization profile of
each reference NAb is the average of five independent experiments. Neutralization
of each HCVpp in each experiment was measured in duplicate. Values are the
rank order of sensitivity of each HCVpp to each reference NAb.

| strain | HC84.26 | HC-1 | CBH-2 | AR1A | AR3A | AR4A | HEPC74 | HEPC98 |
|---|---|---|---|---|---|---|---|---|
| 1b34 | 2 | 3.8 | 4 | 15.6 | 6.4 | 7.4 | 5.6 | 16 |
| 1a53 | 6.2 | 11 | 10.2 | 5.4 | 2.6 | 2.8 | 2.2 | 4.2 |
| 1a31 | 11.4 | 1.2 | 14 | 1.6 | 2.2 | 8.4 | 14 | 12.2 |
| 1b38 | 8.8 | 17.2 | 2.8 | 16 | 5 | 10.6 | 4 | 9 |
| 1a154 | 3.8 | 4 | 15.2 | 3.6 | 6.2 | 4.4 | 7.2 | 1.2 |
| 1b09 | 2 | 9.2 | 1.2 | 16.2 | 1.4 | 3.8 | 1.4 | 13.2 |
| 1a123 | 4.4 | 13.8 | 16.6 | 4.4 | 16.2 | 2.6 | 11.2 | 14 |
| 1a80 | 17.4 | 7 | 13.8 | 9 | 11.2 | 11 | 12.2 | 3.2 |
| 1a157 | 14.8 | 5.6 | 7.4 | 10.6 | 8.8 | 15.2 | 11.2 | 1.8 |
| 1b21 | 7.2 | 5 | 15.4 | 8 | 15 | 2.6 | 2.8 | 11.8 |
| 1a09 | 16.2 | 8.2 | 14.8 | 5.8 | 11.8 | 16.2 | 12.6 | 15.4 |
| 1b58 | 12 | 15.2 | 9.4 | 14.8 | 17.2 | 15 | 14.2 | 11 |
| 1a72 | 15 | 15.2 | 8.8 | 6.6 | 10.2 | 9 | 13 | 12 |
| 1a38 | 8.8 | 12.8 | 6.4 | 13 | 11.2 | 17.4 | 12.4 | 6.2 |
| 1a116 | 16 | 3.4 | 13.8 | 10.2 | 16.8 | 13.4 | 19 | 4.6 |
| 1a142 | 19 | 14.8 | 16 | 7.4 | 12.8 | 6.8 | 12.4 | 12.2 |
| 1b52 | 7.2 | 15.2 | 5.4 | 16.2 | 10 | 15.2 | 9 | 14 |
| 1b14 | 4.6 | 14.2 | 5 | 12.4 | 8.8 | 10.8 | 7.8 | 13.8 |
| 1a129 | 13.2 | 13.2 | 9.8 | 13.2 | 16.2 | 17.4 | 17.8 | 14.2 |

TABLE 2
Neutralization profiles of plasma samples. The neutralization profile of each plasma sample
is the average of two independent experiments, with plasma at 1:100 or 1:20 dilution.
Neutralization of each HCVpp in each experiment was measured in duplicate. Values are
the rank order of sensitivity of each HCVpp to each plasma sample. Rank order for each
sample has been reversed to satisfy input requirements of the deconvolution code.

| strain | P138 | P142 | P150 | P151 | P154 | P49 | P155 | P51 | P52 | P156 | P157 | P158 |
|---|---|---|---|---|---|---|---|---|---|---|---|---|
| 1b34 | 15 | 12 | 15 | 16 | 15 | 1 | 10 | 3 | 17 | 17 | 12 | 16 |
| 1a53 | 3 | 9 | 3 | 6 | 13 | 16 | 9 | 10 | 12 | 8 | 15 | 17 |
| 1a31 | 10.5 | 11.5 | 12 | 12 | 12.5 | 6 | 8.5 | 1 | 12 | 11.5 | 6 | 8.5 |
| 1b38 | 17 | 16 | 12 | 14 | 12 | 17 | 12 | 4 | 15 | 13 | 11 | 12 |
| 1a154 | 10.5 | 6.5 | 12 | 10 | 11.5 | 7.5 | 7 | 8 | 10.5 | 11.5 | 7 | 6.5 |
| 1b09 | 12.5 | 13.5 | 10 | 11.5 | 13 | 11 | 10.5 | 8.5 | 11.5 | 9 | 10.5 | 9.5 |
| 1a123 | 7 | 11 | 10 | 10 | 9 | 13 | 17 | 7 | 10 | 5 | 14 | 3 |
| 1a80 | 2 | 5 | 5 | 8 | 5 | 15 | 11 | 13 | 4 | 15 | 7 | 10 |
| 1a157 | 8 | 10 | 6 | 9 | 8 | 5.5 | 6 | 12.5 | 8 | 7.5 | 6.5 | 10 |
| 1b21 | 10 | 5 | 11 | 11 | 11 | 10 | 11 | 10 | 11 | 10 | 11 | 9 |
| 1a09 | 6.5 | 6 | 5.5 | 8 | 7 | 3 | 6 | 9.5 | 4.5 | 8 | 5 | 7 |
| 1b58 | 12 | 10 | 14 | 4 | 2 | 12 | 8 | 12 | 1 | 6 | 9 | 2 |
| 1a72 | 5 | 3.5 | 4.5 | 3.5 | 4.5 | 4.5 | 3 | 3.5 | 3 | 2.5 | 10.5 | 9.5 |
| 1a38 | 3 | 2 | 5 | 2.5 | 6 | 7 | 9 | 7 | 4.5 | 2.5 | 2 | 3.5 |
| 1a116 | 11 | 11 | 10 | 10 | 7 | 11 | 7 | 7 | 9 | 11 | 10 | 10 |
| 1a142 | 1 | 7 | 13 | 5 | 4 | 8 | 2 | 6 | 9 | 11 | 16 | 14 |
| 1b52 | 7 | 8 | 2.5 | 5.5 | 2 | 6 | 8.5 | 7 | 5.5 | 2.5 | 3.5 | 1.5 |
| 1b14 | 6 | 3 | 9 | 1 | 1 | 9 | 1 | 17 | 6 | 7 | 1 | 7 |
| 1a129 | 4.5 | 4 | 1 | 5 | 5.5 | 3 | 7 | 8 | 3 | 3 | 5.5 | 3.5 |
| strain | P53 | P54 | P161 | P164 | P56 | P168 | P31 | P173 | P175 | P111 | P115 | P113 |
| 1b34 | 12 | 17 | 14 | 15 | 10 | 17 | 8 | 15 | 7 | 12 | 13 | 16 |
| 1a53 | 15 | 8 | 4 | 11 | 14 | 15 | 17 | 11 | 16 | 2 | 10 | 8 |
| 1a31 | 12.5 | 10.5 | 8 | 9.5 | 4.5 | 7 | 10 | 7 | 2 | 2 | 10.5 | 2 |
| 1b38 | 2 | 15 | 8 | 14 | 9 | 14 | 12 | 14 | 13 | 6 | 15 | 5 |
| 1a154 | 9 | 8.5 | 7.5 | 11 | 7.5 | 6 | 9 | 5 | 6 | 12 | 9 | 7 |
| 1b09 | 4 | 13.5 | 10 | 12.5 | 11.5 | 12.5 | 7.5 | 12.5 | 9 | 9 | 14 | 11.5 |
| 1a123 | 7 | 7 | 1 | 3 | 4 | 5 | 9 | 7 | 1 | 5 | 6 | 3 |
| 1a80 | 14 | 4 | 13 | 7 | 6 | 9 | 16 | 12 | 12 | 16 | 7 | 13 |
| 1a157 | 7 | 9 | 9.5 | 10 | 6 | 7 | 7 | 11 | 6 | 10 | 6 | 7.5 |
| 1b21 | 10 | 10 | 11 | 11 | 11 | 11 | 9 | 10 | 11 | 10 | 9 | 11 |
| 1a09 | 10.5 | 3.5 | 7 | 3 | 9.5 | 4.5 | 6 | 5 | 8 | 7.5 | 10 | 9 |
| 1b58 | 10 | 6 | 7 | 5 | 11 | 13 | 6 | 5 | 15 | 9 | 3 | 12 |
| 1a72 | 2.5 | 2 | 3 | 5.5 | 2.5 | 8.5 | 3.5 | 4.5 | 7.5 | 4 | 8.5 | 4.5 |
| 1a38 | 3.5 | 1.5 | 4.5 | 3.5 | 9.5 | 4 | 2.5 | 4 | 7.5 | 4.5 | 2 | 6.5 |
| 1a116 | 11 | 9 | 9 | 9 | 9 | 10 | 6 | 11 | 8 | 11 | 10 | 9 |
| 1a142 | 11 | 11 | 11 | 2 | 1 | 7 | 10 | 3 | 4 | 17 | 12 | 10 |
| 1b52 | 4 | 7.5 | 8.5 | 6 | 10 | 2.5 | 2.5 | 3.5 | 4.5 | 5 | 3.5 | 11 |
| 1b14 | 5 | 13 | 17 | 4 | 17 | 2 | 15 | 13 | 8 | 13 | 5 | 7 |
| 1a129 | 8 | 3.5 | 4 | 8 | 2.5 | 6 | 7.5 | 6.5 | 11.5 | 5 | 1 | 3.5 |
| strain | P461 | P551 | P13 | P14 | P15 | P42 | P126 | P127 | P16 | P41 | P17 | P20 |
| 1b34 | 13 | 17 | 3 | 12 | 16 | 13 | 11 | 15 | 7 | 16 | 3 | 15 |
| 1a53 | 16 | 13 | 14 | 15 | 9 | 2 | 10 | 11 | 16 | 9 | 16 | 17 |
| 1a31 | 9.5 | 4.5 | 4 | 10.5 | 1 | 9 | 12 | 11.5 | 2 | 2.5 | 1 | 11.5 |
| 1b38 | 12 | 12 | 5 | 2 | 8 | 7 | 1 | 14 | 13 | 8 | 4 | 7 |
| 1a154 | 13.5 | 9.5 | 10.5 | 9 | 11.5 | 8 | 13 | 10.5 | 6 | 10 | 9.5 | 11.5 |
| 1b09 | 7.5 | 12.5 | 8.5 | 4.5 | 13 | 9 | 11 | 13.5 | 9 | 13.5 | 7 | 9.5 |
| 1a123 | 14 | 6 | 4 | 7 | 2 | 3 | 12 | 2 | 1 | 2 | 2 | 9 |
| 1a80 | 6 | 1 | 2 | 14 | 15 | 12 | 7 | 6 | 12 | 15 | 13 | 11 |
| 1a157 | 7 | 8 | 10.5 | 7.5 | 10.5 | 11.5 | 9.5 | 9 | 6.5 | 8.5 | 7.5 | 8 |
| 1b21 | 11 | 11 | 8 | 11 | 10 | 10 | 9 | 11 | 11 | 11 | 11 | 11 |
| 1a09 | 4 | 5 | 7 | 8.5 | 6.5 | 5.5 | 2.5 | 5 | 8.5 | 5.5 | 6.5 | 3 |
| 1b58 | 4 | 14 | 6 | 10 | 5 | 8 | 5 | 4 | 15 | 5 | 14 | 12 |
| 1a72 | 11 | 5 | 11.5 | 4.5 | 3.5 | 4.5 | 7 | 6 | 7 | 5 | 6 | 3 |
| 1a38 | 2.5 | 6 | 6 | 4 | 4 | 3.5 | 7.5 | 4 | 8 | 5.5 | 9 | 3.5 |
| 1a116 | 9 | 10 | 10 | 10 | 8 | 7 | 11 | 9 | 6 | 9 | 7 | 6 |
| 1a142 | 8 | 3 | 11 | 11 | 11 | 6 | 6 | 1 | 4 | 11 | 10 | 14 |
| 1b52 | 3 | 9 | 3.5 | 3.5 | 4.5 | 9 | 2.5 | 6 | 6.5 | 5 | 8.5 | 3 |
| 1b14 | 2 | 10 | 13 | 5 | 7 | 15 | 3 | 3 | 8 | 7 | 8 | 4 |
| 1a129 | 4 | 1.5 | 10 | 9 | 9.5 | 8 | 7 | 6 | 9.5 | 7.5 | 10.5 | 3.5 |
| strain | P21 | P22 | P65 | P28 | P29 | P30 | C143 | C47 | C48 | C110 | C55 | C57 |
| 1b34 | 5 | 11 | 12 | 16 | 16 | 17 | 16 | 4 | 12 | 14 | 17 | 15 |
| 1a53 | 4 | 6 | 11 | 17 | 9 | 11 | 14 | 16 | 16 | 13 | 14 | 6 |
| 1a31 | 7.5 | 2 | 12 | 10 | 4 | 9 | 17 | 6 | 12 | 7.5 | 11.5 | 4.5 |
| 1b38 | 3 | 7 | 10 | 13 | 14 | 14 | 10 | 10 | 13 | 12 | 15 | 13 |
| 1a154 | 6 | 11.5 | 12 | 7 | 7.5 | 7 | 13 | 7.5 | 11.5 | 11.5 | 9 | 11 |
| 1b09 | 11 | 5 | 11.5 | 7 | 10.5 | 6 | 15 | 8 | 12 | 13.5 | 10.5 | 12.5 |
| 1a123 | 1 | 1 | 15 | 2 | 2 | 9 | 12 | 8 | 10 | 10 | 7 | 9 |
| 1a80 | 12 | 14 | 4 | 9 | 17 | 15 | 7 | 15 | 8 | 17 | 10 | 16 |
| 1a157 | 9.5 | 8 | 6 | 6 | 6 | 4.5 | 11 | 10.5 | 8.5 | 6 | 5 | 7 |
| 1b21 | 9 | 11 | 10 | 11 | 8 | 8 | | 11 | 11 | 9 | 11 | 11 |
| 1a09 | 10.5 | 7 | 4.5 | 6 | 2.5 | 3 | 1 | 3.5 | 3.5 | 7 | 8.5 | 4.5 |
| 1b58 | 15 | 9 | 3 | 4 | 7 | 2 | 8 | 17 | 6 | 4 | 8 | 4 |
| 1a72 | 3.5 | 5.5 | 3.5 | 7.5 | 8.5 | 10.5 | 9 | 9 | 5.5 | 2 | 3 | 2 |
| 1a38 | 6.5 | 4 | 3.5 | 5 | 3.5 | 4.5 | 3 | 2 | 1.5 | 4.5 | 2.5 | 7.5 |
| 1a116 | 5 | 10 | 11 | 8 | 11 | 11 | | 6 | 9 | 10 | 10 | 9 |
| 1a142 | 10 | 3 | 9 | 14 | 13 | 10 | 6 | 12 | 1 | 2 | 11 | 14 |
| 1b52 | 10 | 12.5 | 9.5 | 3.5 | 4.5 | 3 | 4 | 3 | 4.5 | 4.5 | 4 | 4.5 |
| 1b14 | 13 | 17 | 1 | 3 | 11 | 7 | 5 | 6 | 7 | 11 | 6 | 11 |
| 1a129 | 6.5 | 9.5 | 4 | 9 | 8.5 | 10 | 2 | 7.5 | 4 | 2 | 1 | 2 |
| strain | C117 | C172 | C112 | C176 | C116 | C180 | C181 | C403 | C430 | C18 | C19 | C133 |
| 1b34 | 17 | 16 | 2 | 17 | 17 | 4 | 3 | 1 | 13 | 1 | 5 | 14 |
| 1a53 | 16 | 12 | 1 | 8 | 13 | 1 | 14 | 4 | 10 | 15 | 16 | 16 |
| 1a31 | 6 | 9 | 9 | 3 | 5 | 1.5 | 2.5 | 4 | 12 | 11 | 9.5 | 10.5 |
| 1b38 | 13 | 15 | 16 | 14 | 15 | 16 | 12 | 17 | 12 | 17 | 13 | 11 |
| 1a154 | 12 | 10.5 | 12.5 | 9.5 | 11.5 | 11 | 8 | 12 | 12 | 11 | 14 | 8.5 |
| 1b09 | 12 | 13 | 10.5 | 11.5 | 7 | 10 | 9 | 11.5 | 12 | 10 | 8.5 | 7 |
| 1a123 | 8 | 8 | 4 | 5 | 8 | 5 | 1 | 12 | 3 | 8 | 12 | 12 |
| 1a80 | 11 | 6 | 5 | 16 | 16 | 17 | 17 | 9 | 5 | 3 | 6 | 1 |
| 1a157 | 9 | 4 | 11 | 4.5 | 5.5 | 6.5 | 8 | 5 | 9 | 8.5 | 11 | 9 |
| 1b21 | 11 | 11 | 11 | 11 | 11 | 11 | 10 | 11 | 11 | 10 | 10 | 10 |
| 1a09 | 7 | 4.5 | 5.5 | 6 | 4.5 | 6 | 6.5 | 8.5 | 4 | 3.5 | 6.5 | 5.5 |
| 1b58 | 1 | 7 | 6 | 12 | 12 | 14 | 10 | 5 | 8 | 5 | 1 | 5 |
| 1a72 | 4.5 | 5.5 | 6 | 3 | 6.5 | 6 | 3 | 6.5 | 3.5 | 9.5 | 5.5 | 9.5 |
| 1a38 | 3.5 | 2.5 | 7 | 5 | 5 | 6 | 5 | 6.5 | 6 | 5.5 | 4 | 2.5 |
| 1a116 | 7 | 10 | 7 | 10 | 10 | 8 | 9 | 9 | 10 | 11 | 4 | 11 |
| 1a142 | 12 | 4 | 10 | 11 | 11 | 15 | 15 | 16 | 11 | 11 | 8 | 2 |
| 1b52 | 2 | 8.5 | 7 | 6 | 2 | 4.5 | 5.5 | 8.5 | 6 | 3.5 | 6 | 7 |
| 1b14 | 7 | 9 | 13 | 13 | 7 | 13 | 13 | 6 | 2 | 2 | 4 | 9 |
| 1a129 | 2 | 3 | 3.5 | 2.5 | 2.5 | 6 | 10 | 2 | 2.5 | 5.5 | 5 | 4.5 |

| strain | C24 | C26 | C27 |
|---|---|---|---|
| 1b34 | 16 | 17 | 17 |
| 1a53 | 7 | 13 | 1 |
| 1a31 | 11.5 | 10 | 6 |
| 1b38 | 9 | 16 | 16 |
| 1a154 | 13 | 5.5 | 11.5 |
| 1b09 | 13.5 | 10 | 3.5 |
| 1a123 | 8 | 10 | 7 |
| 1a80 | 10 | 7 | 6 |
| 1a157 | 7 | 3 | 3.5 |
| 1b21 | 1 | 10 | 9 |
| 1a09 | 8.5 | 6 | 3 |
| 1b58 | 3 | 1 | 14 |
| 1a72 | 4.5 | 6.5 | 8 |
| 1a38 | 3.5 | 6 | 8.5 |
| 1a116 | 3 | 11 | 10 |
| 1a142 | 6 | 15 | 13 |
| 1b52 | 9 | 7 | 9 |
| 1b14 | 5 | 5 | 15 |
| 1a129 | 5 | 3 | 2.5 |

### REFERENCES

1. L. Gravitz, Introduction: a smouldering public-health crisis. Nature 474, S2-4 571 (2011).
2. S. D. Holmberg, P. R. Spradling, A. C. Moorman, M. M. Denniston, Hepatitis C in the United States. N. Engl. J Med. 368, 1859-1861 (2013).
3. O. Falade-Nwulia, M. Sulkowski, The HCV care continuum does not end with cure: A call to arms for the prevention of reinfection. J. Hepatol. 66, 267-269 (2017).
4. H. Midgard et al., Hepatitis C reinfection after sustained virological response. J Hepatol. 64, 1020-1026 (2016).
5. T. C. Martin et al., Hepatitis C virus reinfection incidence and treatment outcome among HIV-positive MSM. AIDS 27, 2551-2557 (2013).
6. J. A. Pineda et al., Hepatitis C virus reinfection after sustained virological response in HIV-infected patients with chronic hepatitis C. J. Infect. 71, 571-577 (2015).
7. M. Martinello et al., HCV reinfection incidence among individuals treated for recent infection. J. Viral Hepat. 24, 359-370 (2017).
8. M. M. Mina et al., Resistance to hepatitis C virus: potential genetic and immunological determinants. Lancet Infect. Dis. 15, 451-460 (2015).
9. J. R. Bailey, E. Barnes, A. L. Cox, Approaches, Progress, and Challenges to Hepatitis C Vaccine Development. Gastroenterology 156, 418-430 (2019).
10. A. L. Cox et al., Prospective evaluation of community-acquired acute-phase hepatitis C virus infection. Clin. Infect. Dis. 40, 951-958 (2005).
11. W. O. Osburn et al., Clearance of hepatitis C infection is associated with the early appearance of broad neutralizing antibody responses. Hepatology 59, 2140-2151 (2014).
12. J. M. Pestka et al., Rapid induction of virus-neutralizing antibodies and viral clearance in a single-source outbreak of hepatitis C. Proc. Natl. Acad. Sci. U.S.A 104, 6025-6030 (2007)
13. J. R. Bailey et al., Broadly neutralizing antibodies with few somatic mutations and hepatitis C virus clearance. JCI Insight 2, (2017).
14. S. J. Merat et al., Hepatitis C virus Broadly Neutralizing Monoclonal Antibodies Isolated 25 Years after Spontaneous Clearance. PLoS One 11, e0165047 (2016).
15. A. I. Flyak et al., HCV Broadly Neutralizing Antibodies Use a CDRH3 Disulfide Motif to Recognize an E2 Glycoprotein Site that Can Be Targeted for Vaccine Design. Cell Host Microbe 24, 703-716 e703 (2018).
16. M. D. Colbert et al., Broadly neutralizing antibodies targeting new sites of vulnerability in hepatitis C virus E1E2. J. Virol., (2019).
17. Z. Y. Keck et al., Broadly neutralizing antibodies from an individual that naturally cleared multiple hepatitis C virus infections uncover molecular determinants for E2 targeting and vaccine design. PLoS Pathog. 15, e1007772 (2019).
18. V. J. Kinchen et al., Broadly Neutralizing Antibody Mediated Clearance of 612 Human Hepatitis C Virus Infection. Cell Host Microbe 24, 717-730 e715 (2018).
19. M. Law et al., Broadly neutralizing antibodies protect against hepatitis C virus quasispecies challenge. Nat. Med. 14, 25-27 (2008).
20. Z. Y. Keck et al., Human monoclonal antibodies to a novel cluster of conformational epitopes on HCV e2 with resistance to neutralization escape in a genotype 2a isolate. PLoS. Pathog. 8, e1002653 (2012).
21. J. A. Wong et al., Recombinant hepatitis C virus envelope glycoprotein vaccine elicits antibodies targeting multiple epitopes on the envelope glycoproteins associated with broad cross-neutralization. J. Virol. 88, 14278-14288 (2014).
22. M. C. Sabo et al., Neutralizing monoclonal antibodies against hepatitis C virus E2 protein bind discontinuous epitopes and inhibit infection at a postattachment step. J. Virol. 85, 7005-7019 (2011).
23. P. Zhang et al., Depletion of interfering antibodies in chronic hepatitis C patients and vaccinated chimpanzees reveals broad cross-genotype neutralizing activity. Proc. Natl. Acad. Sci. U.S.A 106, 7537-7541 (2009).
24. A. Kachko et al., Antibodies to an interfering epitope in hepatitis C virus E2 can mask vaccine-induced neutralizing activity. Hepatology 62, 1670-1682 (2015).
25. L. Deng et al., Structural evidence for a bifurcated mode of action in the antibody-mediated neutralization of hepatitis C virus. Proc. Natl. Acad. Sci. U.S.A 110, 7418-7422 (2013).
26. Z. Keck et al., Cooperativity in virus neutralization by human monoclonal antibodies to two adjacent regions located at the amino terminus of hepatitis C virus E2 glycoprotein. J. Virol. 87, 37-51 (2013).
27. Z. Y. Keck et al., Antibody Response to Hypervariable Region 1 Interferes with Broadly Neutralizing Antibodies to Hepatitis C Virus. J. Virol. 90, 3112-3122 (2016).
28. J. R. Bailey et al., Naturally selected hepatitis C virus polymorphisms confer broad neutralizing antibody resistance. J Clin. Invest. 125, 437-447 (2015).
29. S. Munshaw et al., Computational reconstruction of bolela, a representative synthetic hepatitis C virus subtype 1a genome. J. Virol. 86, 5915-5921 (2012).
30. V. J. Kinchen, J. R. Bailey, Defining Breadth of Hepatitis C Virus Neutralization. Front. Immunol. 9, 1703 (2018).
31. I. S. Georgiev et al., Delineating antibody recognition in polyclonal sera from patterns of HIV-1 isolate neutralization. Science 340, 751-756 (2013).
32. E. Giang et al., Human broadly neutralizing antibodies to the envelope glycoprotein complex of hepatitis C virus. Proc. Natl. Acad. Sci. U.S.A 109, 6205-648 (2012).
33. A. M. Owsianka et al., Broadly neutralizing human monoclonal antibodies to the hepatitis C virus E2 glycoprotein. J. Gen. Virol. 89, 653-659 (2008).
34. V. J. Kinchen, A. L. Cox, J. R. Bailey, Can Broadly Neutralizing Monoclonal Antibodies Lead to a Hepatitis C Virus Vaccine? Trends Microbiol., (2018).
35. N. Tzarum et al., Genetic and structural insights into broad neutralization of hepatitis C virus by human VH1-69 antibodies. Sci Adv 5, eaav1882 (2019).
36. R. Gopal et al., Probing the antigenicity of hepatitis C virus envelope glycoprotein complex by high-throughput mutagenesis. PLoS Pathog. 13, 657 e1006735 (2017).
37. B. G. Pierce et al., Global mapping of antibody recognition of the hepatitis C virus E2 glycoprotein: Implications for vaccine design. Proc. Natl. Acad. Sci. U.S.A., (2016).
38. R. El-Diwany et al., Extra-epitopic hepatitis C virus polymorphisms confer resistance to broadly neutralizing antibodies by modulating binding to scavenger receptor B1. PLoS Pathog. 13, e1006235 (2017).
39. L. N. Wasilewski et al., A Hepatitis C Virus Envelope Polymorphism Confers Resistance to Neutralization by Polyclonal Sera and Broadly Neutralizing Monoclonal Antibodies. J Virol. 90, 3773-3782 (2016).
40. J. Prentoe et al., Hypervariable region 1 and N-linked glycans of hepatitis C regulate virion neutralization by modulating envelope conformations. Proc. Natl. Acad. Sci. U.S.A. 116, 10039-10047 (2019).
41. M. C. Mankowski et al., Synergistic anti-HCV broadly neutralizing human monoclonal antibodies with independent mechanisms. Proc. Natl. Acad. Sci. U.S.A., (2017).
42. T. H. Carlsen et al., Breadth of neutralization and synergy of clinically relevant human monoclonal antibodies against HCV genotypes 1a, 1b, 2a, 2b, 2c, and 3a. Hepatology 60, 1551-1562 (2014).
43. M. C. Mankowski et al., Synergistic anti-HCV broadly neutralizing human monoclonal antibodies with independent mechanisms. Proc. Natl. Acad. Sci. U S. A. 115, E82-E91 (2018).
44. Y. P. de Jong et al., Broadly neutralizing antibodies abrogate established hepatitis C virus infection. Sci. Transl. Med. 6, 254ra129 (2014).
45. M. Law et al., Broadly neutralizing antibodies protect against hepatitis C virus quasispecies challenge. Nat. Med. 14, 25-27 (2008).
46. S. E. Frey et al., Safety and immunogenicity of HCV E1E2 vaccine adjuvanted with MF59 administered to healthy adults. Vaccine 28, 6367-6373 (2010).
47. T. J. Liang, Current progress in development of hepatitis C virus vaccines. Nat. Med. 19, 869-878 (2013).
48. G. J. Tobin et al., Deceptive imprinting and immune refocusing in vaccine design. Vaccine 26, 6189-6199 (2008).
49. A. W. Chung, G. Alter, Systems serology: profiling vaccine induced humoral immunity against HIV. Retrovirology 14, 57 (2017).
50. K. G. Hadlock et al., Human monoclonal antibodies that inhibit binding of hepatitis C virus E2 protein to CD81 and recognize conserved conformational epitopes. J Virol. 74, 10407-10416 (2000).
51. M. Hsu et al., Hepatitis C virus glycoproteins mediate pH-dependent cell entry of pseudotyped retroviral particles. Proc. Natl. Acad. Sci. U.S.A 100, 7271-7276 (2003).
52. C. Logvinoff et al., Neutralizing antibody response during acute and chronic hepatitis C virus infection. Proc. Natl. Acad. Sci. U.S.A 101, 10149-10154 (2004).
53. L. Wasilewski, S. Ray, J. R. Bailey, Hepatitis C virus resistance to broadly neutralizing antibodies measured using replication competent virus and pseudoparticles. J Gen. Virol., (2016).
54. J. R. Bailey, R. A. Urbanowicz, J. K. Ball, M. Law, S. K. H. Foung, Standardized Method for the Study of Antibody Neutralization of HCV Pseudoparticles (HCVpp). Methods Mol. Biol. 1911, 441-450 (2019).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A method of identifying Hepatitis C virus (HCV) neutralizing antibodies, comprising:
obtaining a biological sample from a subject having been infected with HCV;
measuring neutralization of HCV pseudoparticles (HCVpp) or replication competent HCV (HCVcc) by antibodies specific for an HCV in the biological sample;
generating a neutralization profile of each biological sample;
deconvoluting the HCV-specific neutralizing antibodies by generating a reference antibody neutralization profile;
correlating the reference antibody neutralization profile to the biological sample's neutralization profile; and,
identifying the HCV neutralizing antibodies.

2. The method of claim 1, wherein the biological sample is whole blood, lymphocytes, serum, or plasma.

3. The method of claim 1, wherein the neutralization profile comprises a ranking of relative neutralization of each HCVpp by each reference antibody or biological sample.

4. The method of claim 1, wherein the reference antibody neutralization profiles were added in various proportions to generate an array of possible combined antibody neutralization profiles.

5. The method of claim 1, wherein a specific combined reference antibody neutralization profile is correlated with each plasma neutralization profile to identify the proportion of each reference antibody contributing to the neutralization profile of the biological sample.

6. The method of claim 1, further comprising identifying HCV epitope specificities for each neutralizing antibody.

7. The method of claim 1, wherein the neutralization profiles identify individual antibodies which bind to distinct HCV epitopes or are cross-reactive to related HCV epitopes.

8. The method of claim 1, further comprising isolating the HCV neutralizing antibodies.

9. The method of claim 1, wherein the method comprises a high throughput format.

10. A vaccine comprising a polypeptide having an Hepatitis C virus (HCV) epitope which induces an HCV neutralizing antibody, the antibody identified by a method of claim 1.

11. An isolated hybrid cell producing a Hepatitis C virus (HCV) neutralizing antibody identified by a method of claim 1.

12. A method of treating a subject infected with a Hepatitis C virus (HCV), comprising administering to the subject a therapeutically effective amount of HCV neutralizing antibodies identified by a method of claim 1.

13. A method of identifying virus-specific neutralizing antibodies comprising,
obtaining a biological sample from a subject having been infected with a virus;
measuring neutralization of HCVpp or HCVcc by the biological sample;
generating a neutralization profile of each biological sample;
deconvoluting the virus-specific neutralizing antibodies by generating a reference antibody neutralization profile;
correlating the reference antibody neutralization profile to the biological sample's neutralization profile; and,
identifying the virus neutralizing antibodies.

14. The method of claim 13, wherein the virus comprises: adenoviruses, arenaviruses, bunyaviruses, flaviviruses, filoviruses, herpesviruses, noroviruses, orthomyxoviruses, poxviruses, papilloma viruses, paramyxoviruses, reoviruses, rhabdoviruses, retroviruses, or togaviruses.

15. A method of treating a subject infected with a Hepatitis C virus (HCV), comprising administering to the subject a therapeutically effective amount of HCV neutralizing antibodies identified by a vaccine of claim 10.
